(19) United States
(12) Patent Application Publication
(10) Pub. No.: US 2010/0145002 A1
(43) Pub. Date: Jun. 10, 2010

(54) TETRACARBOXYLIC ACID OR POLYESTERIMIDE THEREOF, AND PROCESS FOR PRODUCTION OF THE SAME

(75) Inventors: Masatoshi Hasegawa, Chiba (JP); Haruhiko Kusaka, Kanagawa (JP); Jun Enda, Kanagawa (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Mitsubishi Chemical Corporation, Minato-ku, Tokyo (JP)

(21) Appl. No.: 11/916,299

(22) PCT Filed: Jun. 1, 2006
(86) PCT No.: PCT/JP2006/311026

§ 371 Date: Mar. 25, 2008

(30) Foreign Application Priority Data
Jun. 1, 2005 (JP) 2005-161490
Sep. 13, 2005 (JP) 2005-264852
Mar. 23, 2006 (JP) 2006-081058

(51) Int. Cl. C08G 73/10; C07D 307/77; C07C 69/74; C08G 69/26

(52) U.S. Cl. 528/310; 549/242; 560/127; 549/244; 528/353

(57) ABSTRACT

The present invention provides a useful and novel alicyclic polyesterimide.

An alicyclic polyesterimide produced by imidation of an alicyclic polyesterimide precursor is found to be a useful material in industrial fields, the alicyclic polyesterimide precursor being obtained by reacting an alicyclic tetracarboxylic anhydride having an ester group or a class of tetracarboxylic acid thereof as a starting material with an amine.

### TECHNICAL FIELD

The present invention relates to an alicyclic tetracarboxylic anhydride containing an ester group or a class of tetracarboxylic acid thereof, an alicyclic polyesterimide precursor and alicyclic polyesterimide produced starting from the same, and a process for producing the same.

### BACKGROUND ART

Since polyimides have properties such as not only excellent thermal resistance but also chemical resistance, radiation resistance, electric insulation, and excellent mechanical properties in combination, they have currently widely utilized in various electronic devices such as flexible printed wiring circuit boards, substrates for tape automation bonding, protective films of semiconductor elements, and interlayer insulating films for integrated circuits. The polyimides are also very useful materials in view of convenience of production processes, a high film purity, and easiness of improving physical properties, and thus recently, material designs for functional polyimides suitable for various applications have been performed.

Since most of polyimides are insoluble in organic solvents and are not melted even when heated at a temperature higher than glass transition temperature, it is usually not easy to mold and process the polyimides themselves. Therefore, a polyimide is generally formed as a film by reacting an aromatic tetracarboxylic dianhydride such as pyromellitic anhydride with an aromatic diamine such as diaminodiphenyl ether in an equimolar amount in an aprotic polar organic solvent such as dimethylacetamide to polymerize into a polyimide precursor having a high polymerization degree, forming the solution into a film or the like, and heating it at a temperature of about 250° C. to 350° C. to effect dehydrative ring-closure (imidation).

The thermal stress generated in the progress of cooling a polyimide/metal substrate laminate from the imidation temperature to room temperature frequently causes severe problems such as curling, film peeling, and cracking. In recent years, with densification of electronic circuits, multilayer wiring boards have been employed. However, even if the cooling does not result in peeling or cracking of the film, residual stress in the multilayer board remarkably lowers reliability of devices, so that it is investigated to reduce the thermal stress. However, there is a problem that a resin having low thermal stress shows low solubility to solvents and thus is poor in operability.

On the other hand, in the case where a polyimide is soluble in an organic solvent, since the heating imidation step is not necessary, it is sufficient to vaporize and dry the solvent at a much lower temperature than the heating imidation temperature after application of an organic solvent solution (varnish) of the polyimide on a metal substrate and thus it is possible to reduce the thermal stress in a metal substrate/insulating film laminate. However, the polyimides which is soluble in an organic solvent and have been in practical use are limited and hence it has been desired to develop a polyimide having a variety of physical properties and soluble in a solvent.

Furthermore, polyimides are known to generally have high water absorbability. Water absorption in an insulating layer causes severe problems such as dimensional change of insulating films and deterioration of electric properties. As a molecular design for realizing low water absorbability, it has been reported to introduce an ester bond into a polyimide skeleton (Non-Patent Document 1).

Moreover, recently, speeding-up of computing speed of microprocessors and shortening of rise time of clock signals become particularly important problems in the information processing and communication fields. For the purpose, it is necessary to lower the dielectric constant of a polyimide film to be used as an insulating film. Also, the case is advantageous for high-density wiring and multilayer board formation for the purpose of shortening of electric wiring length because lower dielectric constant of the insulating film enables reduction of thickness of the insulating layer.

For lowering the dielectric constant of a polyimide, it is effective to introduce a fluorine substituent into the skeleton (Non-Patent Document 2). However, the use of a fluorinated monomer is disadvantageous in view of costs.

In addition, the reduction of it electrons by replacing the aromatic unit with an alicyclic unit is also an effective means for lowering the dielectric constant (Non-Patent Document 3).

However, it is not easy as a molecular design to obtain a polyimide having all of low dielectric constant (3.0 or less as a target value), low water absorbability, and solvent solubility simultaneously and also possessing solder thermal resistance and thus a practical material satisfying such required properties is currently not known. Although a low-dielectric-constant polymer material and inorganic material other than polyimides have been investigated, it is a current situation that the required properties are not satisfied in dielectric constant, thermal resistance, and toughness.

Furthermore, recently, from a demand for development to optical material applications, there is an increasing request for a polyimide showing a high transparency in a visible light region. If a polyimide having thermal resistance, solubility, appropriate toughness in addition to the transparency is obtained, it can be suitably used as a flexible substrate for liquid crystal displays and EL displays and various optical characteristic members to be used inside thereof. However, a material having all of such properties is currently not known.

Moreover, for the purpose of subjecting the polyimide as an insulating layer to though-hole formation and micro-fabrication, a photosensitive polyimide system wherein photosensitivity is imparted to a polyimide or a precursor thereof has been intensively studied. On the other hand, through-hole formation or the like has been performed by etching a polyimide with a basic substance. However, since the etching rate of the polyimide film with an alkali is usually low in the latter, an etching solution is limited to a special basic substance such as ethanolamine and, even when ethanolamine is used, the method cannot be applied to all the polyimides. If a material having the above required properties and capable of being easily etched with a common basic substance is developed, an extremely valuable material in the above industrial fields may be provided but such a material is currently not known.

### Non-Patent Document 1: Kobunshi Toronkai Yokoshu, 53, 4115 (2004)

### Non-Patent Document 2: Macromolecules, 24, 5001 (1991)

### Non-Patent Document 3: Macromolecules, 32, 4933 (1999)

### DISCLOSURE OF THE INVENTION

### Problems that the Invention is to Solve

The present invention provides an alicyclic polyesterimide useful in electronic material fields such as electric insulating films and laminates in various electronic devices and flexible printed wiring boards; display device fields such as substrates for liquid crystal displays, substrates for organic electroluminescent (EL) displays, and substrates for electronic paper; optical material fields such as lenses, diffraction gratings, and light guides; semiconductor fields such as buffer coating films and interlayer insulating films; and substrates for solar cells, photosensitive materials, and the like, since the polyesterimide has all of high glass transition temperature, high transparency, low water absorbability, and etching properties in combination. The invention also provides a precursor thereof, further a novel monomer as a starting material thereof, and a process for producing the same.

### Means for Solving the Problems

As a result of the extensive studies in consideration of the above problems, the present inventors have found that an alicyclic polyesterimide (5) derived from imidation of an alicyclic polyesterimide precursor (4) obtained by reacting an alicyclic tetracarboxylic anhydride having an ester group or a class of tetracarboxylic acid thereof represented by any of the following general formulae (1) to (3) as a starting material with an amine, can be a useful material in the above industrial fields and thus the invention has been accomplished.

Namely, the first gist of the invention lies in an alicyclic tetracarboxylic anhydride having an ester group or a class of tetracarboxylic acid thereof represented by any of the following general formulae (1) to (3):

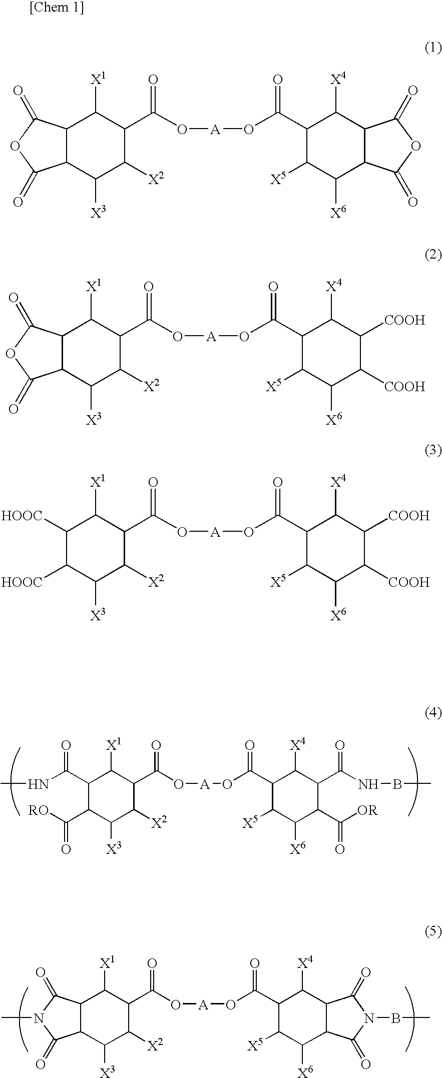

wherein in the formulae (1) to (5), A represents a divalent group; X¹, X², X³, X⁴, X⁵, and X⁶ each independently represents a hydrogen atom, a halogen atom, a nitrile group, a nitro group, an alkyl group, an alkenyl group, an alkynyl group, an alkoxy group, an amino group, or an amide group; B represents a divalent aromatic or aliphatic group; and R represents a hydrogen atom, an alkyl group having 1 to 12 carbon atoms, or a silyl group.

The second gist lies in the above alicyclic tetracarboxylic anhydride having an ester group or a class of tetracarboxylic acid thereof, wherein A in the above formulae (1) to (3) is a divalent group having an aromatic group and/or an aliphatic group.

The third gist lies in the above alicyclic tetracarboxylic anhydride having an ester group or a class of tetracarboxylic acid thereof, wherein in the above formulae (1) to (3), X¹, X², X³, X⁴, X⁵, and X⁶ is a hydrogen atom and A is a structure containing at least one cyclic structure.

The fourth gist lies in a process for producing the above alicyclic tetracarboxylic anhydride having an ester group or a class of tetracarboxylic acid thereof, which comprises: converting an aromatic ring-hydrogenated trimellitic anhydride into an acid halide; and reacting the resulting acid halide with a diol in the presence of a basic substance.

The fifth gist lies in an alicyclic polyesterimide precursor of the above formula (4) derived from the above alicyclic tetracarboxylic anhydride having an ester group of the above formulae (1) to (3) or a class of tetracarboxylic acid thereof and a diamine.

The sixth gist lies in an alicyclic polyesterimide represented by the above formula (5).

The seventh gist lies in a process for producing the alicyclic polyesterimide, which comprises: a cyclizing imidation reaction of the alicyclic tetracarboxylic dianhydride containing an ester group represented by any of the above formulae (1) to (3) with a class of diamine.

The eighth gist lies in a process for producing the alicyclic polyesterimide, wherein the alicyclic polyesterimide precursor represented by the above formula (4) is subjected to the cyclizing imidation reaction.

The ninth gist lies in the process for producing the alicyclic polyesterimide, wherein the cyclizing imidation reaction is carried out using heating and/or a dehydrating reagent at the cyclizing imidation reaction of the alicyclic polyesterimide precursor represented by the above formula (4) and a class of diamine.

The tenth gist lies in a film comprising a resin containing the constitutional unit of the above formula (5).

The eleventh gist lies in a member for liquid crystals using a film produced from the resin containing the constitutional unit of the above formula (5).

### ADVANTAGE OF THE INVENTION

According to the present invention, there can be provided a resin having all of high glass transition temperature, high transparency, high organic solvent solubility, low birefringence, and alkali-etching properties in combination as well as a starting material thereof. Specifically, owing to the bonding of the acid anhydride group onto the cyclohexane ring in the tetracarboxylic dianhydride which is a starting material of the resin according to the invention, enhancement of transparency and decrease in dielectric constant become possible by suppressing n-electron conjugation and intramolecular and intermolecular charge transfer interaction in the polyesterimide. Moreover, the ester bond in the polyesterimide enables alkali-etching in the case where micro-fabrication such as through-hole formation is necessary.

### BRIEF DESCRIPTION OF THE DRAWINGS

FIG. 1 shows an infrared absorption spectrum of the alicyclic tetracarboxylic acid described in Example 1.

FIG. 2 shows an NMR spectrum of the alicyclic tetracarboxylic acid described in Example 1 measured in DMSO.

FIG. 3 shows a differential scanning calorimetric curve of the alicyclic tetracarboxylic acid described in Example 1.

FIG. 4 shows an infrared absorption spectrum of the alicyclic polyesterimide precursor thin film described in Example 2.

FIG. 5 shows an infrared absorption spectrum of the alicyclic polyesterimide thin film described in Example 2.

FIG. 6 shows an infrared absorption spectrum of the alicyclic polyesterimide precursor thin film described in Example 3.

FIG. 7 shows an infrared absorption spectrum of the alicyclic polyesterimide thin film described in Example 3.

FIG. 8 shows an infrared absorption spectrum of the alicyclic polyesterimide precursor thin film described in Example 4.

FIG. 9 shows an infrared absorption spectrum of the alicyclic polyesterimide thin film described in Example 4.

FIG. 10 shows an infrared absorption spectrum of the alicyclic polyesterimide precursor thin film described in Example 5.

FIG. 11 shows an infrared absorption spectrum of the alicyclic polyesterimide thin film described in Example 5.

FIG. 12 shows an infrared absorption spectrum of the alicyclic polyesterimide precursor thin film described in Example 6.

FIG. 13 shows an infrared absorption spectrum of the alicyclic polyesterimide thin film described in Example 6.

FIG. 14 shows an infrared absorption spectrum of the alicyclic polyesterimide thin film described in Example 7.

FIG. 15 shows an infrared absorption spectrum of the alicyclic polyesterimide thin film described in Example 8.

FIG. 16 shows an infrared absorption spectrum of the alicyclic tetracarboxylic acid described in Example 9.

FIG. 17 shows an NMR spectrum of the alicyclic tetracarboxylic acid described in Example 9 measured in DMSO.

FIG. 18 shows an infrared absorption spectrum of the alicyclic polyesterimide thin film described in Example 10.

FIG. 19 shows an infrared absorption spectrum of the alicyclic tetracarboxylic acid described in Example 11.

FIG. 20 shows an infrared absorption spectrum of the alicyclic polyesterimide thin film described in Example 12.

FIG. 21 shows an infrared absorption spectrum of the alicyclic tetracarboxylic acid described in Example 13.

FIG. 22 shows an NMR spectrum of the alicyclic tetracarboxylic acid described in Example 13 measured in DMSO.

FIG. 23 shows a differential scanning calorimetric curve of the alicyclic tetracarboxylic acid described in Example 13.

### BEST MODE FOR CARRYING OUT THE INVENTION

The following will explain the invention in detail but the explanation of the constitutional requirement described in the following is one example (representative example) of the embodiments of the invention and the invention is not limited to the content. The term “a class of” means “a compound of”. For example, a class of tetracarboxylic acid and a class of diamine means a compound of tetracarboxylic acid and a compound of diamine, respectively.

### <Alicyclic Tetracarboxylic Anhydride Containing Ester Group or Class of Tetracarboxylic Acid Thereof>

The alicyclic tetracarboxylic anhydride having an ester group of the invention refers to a compound represented by the following formula (1) wherein the both ends form anhydride and a class of alicyclic tetracarboxylic acid having an ester group refers to a compound represented by the following formula (2) wherein one end forms a condensed ring and the other end is carboxylic acid and a tetracarboxylic acid represented by the following formula (3).

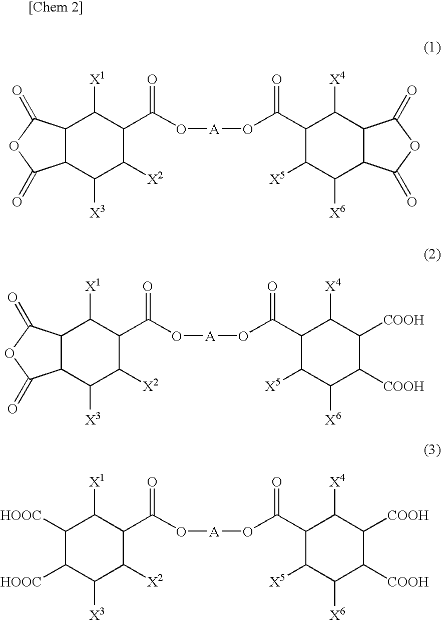

As a structure of A in the above formulae (1) to (3), A is structurally not particularly limited so far as it is bonded at the two sites to the carboxyl groups so as to form each of the above structures.

Specifically, in the formulae (1) to (3), A may be any divalent group. The compound of the invention has a characteristic of a structure having two cyclohexane rings and two ester groups connecting the rings and the structure affords physical properties such as high transparency, high toughness, high solvent solubility at the time when the compound is converted into the alicyclic polyesterimide resin of the invention. Namely, even when the structure of A is any divalent group, these physical properties of the present compound tend to be not remarkably affected. Therefore, the structure of A is not particularly limited so far as it is any divalent group.

Among the divalent groups, preferred is a group having a cyclic structure. The structure having a cyclic structure refers to one containing an aromatic group or an alicyclic structure in A. When A contains a cyclic structure, improvement in thermal resistance and dimensional stability at the time when the compound is converted into the alicyclic polyesterimide resin is provided. Moreover, in the case where A contains an alicyclic structure, there can be obtained a characteristic that light absorption within a UV region can be reduced while thermal resistance is maintained. Examples of specific structure include a phenylene group, a naphthylene group, a biphenylene group, a diphenyl ether group, a diphenyl sulfone group, a 4,4′-(9-fluorenylidene)diphenyl group, a methylenediphenyl group, an isopropylidenediphenyl group, a 3,3′,5,5′-tetramethyl-(1,1′-biphenyl) group, and the like as an aromatic groups, which are all divalent groups, and a cyclohexylene group, a cyclohexanedimethylene group, a decahydronaphthylene group, and the like as alicyclic structures. Furthermore, the structure may be a structure wherein these groups are plurally combined with each other or with the other group(s) via a connecting group. Specific examples of the applicable connecting group include a methylene group (—CH₂—), an ether group (—O—), an ester group (—C(O)O—), a keto group (—C(O)—), a sulfonyl group (—SO₂—), a sulfinyl group (—SO—), a sulfenyl group (—S—), a 9,9-fluorenylidene group, and the like. With regard to the group containing the above divalent cyclic structure, the substitution position is not particularly limited. For example, in the case of a phenylene group, when substituted in a 1,4-position, the structure of -A- becomes linear, so that it is expected that thermal resistance is improved and a linear expansion coefficient decreases and hence the case is preferred. On the other hand, in the case where the phenylene group is substituted in a 1,3-position, improvement of solubility to solvents is expected since the structure of -A- is bent, so that the case is preferred. Therefore, with regard to the substitution position, it is preferred that A having an appropriately suitable structure is selected depending on required physical properties.

As a more preferred structure, A is a group containing an aromatic group. When an aromatic group is contained, thermal stability and dimensional stability are further improved and also improvement of refractive index is achieved when it is converted into the alicyclic polyesterimide resin. As specific ones of the aromatic group, the above-mentioned groups are applicable but, in particular, a phenylene group, a biphenylene group, a diphenyl ether group, a diphenyl sulfone group, a 4,4′-(9-fluorenylidene)diphenyl group, a 3,3′,5,5′-tetramethyl-(1,1′-biphenyl) group and the like are particularly preferred in view of a more rigid structure. Furthermore, a phenylene group, a 4,4′-(9-fluorenylidene)diphenyl group, and a 3,3′,5,5′-tetramethyl-(1,1′-biphenyl) group are preferred in view of availability of the starting material and good physical properties of the resulting resins.

Moreover, X¹, X², X³, X⁴, X⁵, and X⁶ in the above formulae (1) to (3) each independently represents a hydrogen atom, a halogen atom, a nitrile group, a nitro group, an alkyl group, an alkenyl group, an alkynyl group, an alkoxy group, an amino group, or an amide group. The number of carbon atoms of the alkyl group, alkenyl group, alkynyl group, alkoxy group, amino group, or amide group is preferably from 1 to 10. More specifically, examples of the alkyl group include a methyl group, an ethyl group, an n-propyl group, an i-propyl group, an n-butyl group, and the like. Examples of the alkoxy group include a methoxy group, an ethoxy group, an n-propoxy group, an i-propoxy group, an n-butoxy group, and the like. Moreover, examples of the halogen atom include a fluorine atom, a chlorine atom, a bromine atom, and an iodine atom. Among these examples, X¹, X², X³, X⁴, X⁵, and X⁶ in the above formulae (1) to (3) each independently is preferably a hydrogen atom or a halogen atom in view of easy availability of the starting material. In this case, the number of the halogen atoms and the substitution position(s) are not particularly limited. More preferred is a case wherein all of X¹, X², X³, X⁴, X⁵, and X⁶ in the above formulae (1) to (3) are hydrogen atoms.

Preferred structures as combinations of A and X¹, X², X³, X⁴, X⁵, and X⁶ are those wherein A is a group having a cyclic structure and X¹, X², X³, X⁴, X⁵, and X⁶ each is independently composed of a halogen atom or a hydrogen atom. More preferred is one wherein A is a group having a cyclic structure and all of X¹, X², X³, X⁴, X⁵, and X⁶ are composed of hydrogen atoms.

### <Alicyclic Polyesterimide Precursor and Alicyclic Polyesterimide>

The alicyclic polyesterimide precursor and the alicyclic polyesterimide of the invention refer to an alicyclic polyesterimide precursor represented by the following formula (4) and a alicyclic polyesterimide represented by the following formula (5).

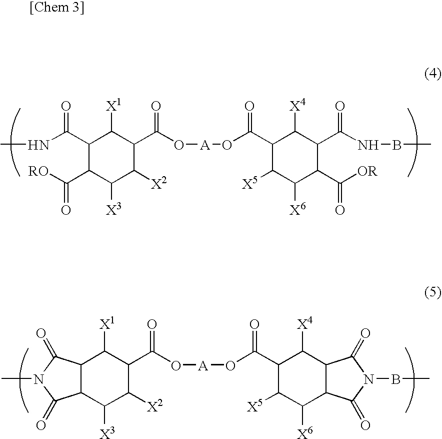

A, X¹, X², X³, X⁴, X⁵, and X⁶ in the above formulae (4) and (5) are the same as described in the article of the alicyclic tetracarboxylic anhydride containing an ester group. In this connection, the bonding positions of the —CONH— group and —COOR group bonded to each cyclohexane ring in the above formula (4) may be interchanged.

B in the formulae (4) and (5) can be any divalent group. The alicyclic polyesterimide precursor (4) and the alicyclic polyesterimide (5) of the invention have a characteristic of a structure having two cyclohexane ring and two ester groups connecting the rings and the structure affords high transparency, high toughness, and solvent solubility. Namely, even when the structure of B is any divalent group, these physical properties of the present compound tend to be not remarkably affected. Therefore, the structure of B is not particularly limited so far as it is any divalent group.

Among the divalent groups, preferred as the structure of B is a group having a cyclic structure. The structure having a cyclic structure refers to one containing an aromatic group or an alicyclic structure in B. When B contains a cyclic structure, improvement in thermal resistance and dimensional stability at the time when the compound is converted into the alicyclic polyesterimide resin is provided. Moreover, in the case where B contains an alicyclic structure, there can be obtained a characteristic that light absorption in a UV region can be reduced while thermal resistance is maintained. Examples of specific structure include a phenylene group, a naphthylene group, a biphenylene group, a diphenyl ether group, a diphenyl sulfone group, a 4,4′-(9-fluorenylidene)diphenyl group, a methylenediphenyl group, an isopropylidenediphenyl group, 3,3′-dimethyl-1,1′-biphenyl group, a 3,3′,5,5′-tetramethyl-1,1′-biphenyl group, 2,2′-bis(trifluoromethyl)-1,1′-biphenyl group, and the like as an aromatic groups, which are all divalent groups, and a cyclohexylene group, a cyclohexanedimethylene group, a dicyclohexyl ether group, a methylenedicyclohexyl group, a decahydronaphthylene group, and the like as alicyclic structures. Furthermore, the structure may be a structure wherein these groups are plurally combined with each other or with the other group(s) via a connecting group. Specific examples of the applicable connecting group include a methylene group (—CH₂—), an ether group (—O—), an ester group (—C(O)O—), a keto group (—C(O)—), a sulfonyl group (—SO₂—), a sulfinyl group (—SO—), a sulfenyl group (—S—), a 9,9-fluorenylidene group, and the like. In this connection, with regard to the group containing the above divalent cyclic structure, the substitution position is not particularly limited. For example, in the case of a phenylene group, when substituted in a 1,4-position, the structure of —B— becomes linear, so that it is expected that thermal resistance is improved and a linear expansion coefficient decreases and hence the case is preferred. On the other hand, in the case where the phenylene group is substituted in a 1,3-position, improvement of solubility to solvents is expected since the structure of —B— is bent, so that the case is preferred. Therefore, with regard to the substitution position, it is preferred that B having an appropriately suitable structure is selected depending on required physical properties.

As a more preferred structure, B is a group containing an aromatic group. When an aromatic group is contained, thermal stability and dimensional stability are further improved and also improvement of refractive index is achieved when it is converted into the alicyclic polyesterimide resin. As specific ones of the aromatic group, the above-mentioned groups are applicable but, in particular, a phenylene group, a biphenylene group, a diphenyl ether group, a diphenyl sulfone group, a 4,4′-(9-fluorenylidene)diphenyl group, a 3,3′,5,5′-tetramethyl-1,1′-biphenyl group, and the like are particularly preferred in view of a more rigid structure.

R represents a hydrogen atom, an alkyl group having 1 to 12 carbon atoms, or a silyl group. Examples of the alkyl group include a methyl group, an ethyl group, an n-propyl group, and an i-propyl group and examples of the silyl group include a trimethylsilyl group, a triethylsilyl group, a dimethyl-t-butylsilyl group as employable examples. In particular, in view of high eliminating ability, a trimethylsilyl group and a dimethyl-t-butylsilyl group are preferred.

Preferred structures as combinations of A and B, X¹, X², X³, X⁴, X⁵, and X⁶ are those wherein A and B each is a group having a cyclic structure and X¹, X², X³, X⁴, X⁵, and X⁶ each is independently composed of a halogen atom or a hydrogen atom. More preferred is one wherein A and B each is a group having a cyclic structure and all of X¹, X², X³, X⁴, X⁵, and X⁶ are composed of hydrogen atoms. In this connection, the structures of A and B on this occasion may be the same or different from each other.

### <Process for Producing Alicyclic Tetracarboxylic Anhydride Having an Ester Group or a Class of Tetracarboxylic Acid Thereof>

The alicyclic tetracarboxylic anhydride having an ester group or a class of tetracarboxylic acid thereof of the invention can be produced using, for example, a trimellitic anhydride whose aromatic ring is hydrogenated (hereinafter, referred to as aromatic ring-hydrogenated trimellitic anhydride) and a diol as starting materials. The following describes a process for producing the same as one example but, in the invention, the production process is not limited so far as it can produce the alicyclic tetracarboxylic anhydride having an ester group or the tetracarboxylic acid thereof having the above-mentioned structure.

The process for producing the aromatic ring-hydrogenated trimellitic anhydride is not particularly limited and any known or used method can be employed. In the case of producing an acid anhydride wherein the cyclohexane ring has a substituent (the case where X¹, X², X³, X⁴, X⁵, and X⁶ in the general formula (1) each is independently different from the hydrogen atom), the process for producing the same is not particularly limited, examples thereof including a process of aromatic ring-hydrogenation using a trimellitic anhydride having a substituent introduced thereinto, a process of introducing a substituent into the aromatic ring-hydrogenated trimellitic anhydride, and the like.

As a specific example of the production process, the aromatic ring-hydrogenated trimellitic anhydride to be a starting material for the alicyclic tetracarboxylic anhydride having an ester group or a class of tetracarboxylic acid thereof can be obtained by hydrogenating trimellitic acid or trimellitic anhydride. Alternatively, it can be also produced by aromatic ring-hydrogenation of an ester of trimellitic acid, then hydrolysis of the ester part, and intramolecular dehydration into acid anhydride. Specifically, U.S. patent application Laid-Open No. U.S. Pat. No. 5,412,108 discloses that it can be produced by aromatic ring-hydrogenation of trimellitic anhydride. In the specification of the U.S. patent application Laid-Open, use of an Rh catalyst wherein Rh metal is supported on a certain specific elemental substance as a hydrogenation catalyst usable for aromatic ring-hydrogenation is advantageous but, in addition to the catalyst, any catalyst can be used without particular limitation so far as it is a catalyst using a metal capable of hydrogenation of aromatic nuclei, such as Pd, Ru, Ni, or Pt. These metal catalysts can be used supported on a support or as a metal alone and further may be used with adding the other component(s) to these metals as needed.

The aromatic ring-hydrogenation usually affords a mixture of four kinds of stereoisomers (8 kinds containing optical isomers) with regard to the three substituents on the cyclohexane ring. These stereoisomers may be used as a mixture as it is in the next reaction or may be used after increasing the concentration of single isomer or a specific isomer by purification such as recrystallization. Moreover, as a method of selectively obtaining a specific isomer, for example, a product wherein the three substituents are all controlled as cis-configuration can be obtained as a main component when the method described in U.S. patent application Laid-Open No. U.S. Pat. No. 5,412,108 or the like is used, for example. In this case, purity of the all-cis isomer is usually 90% or more, preferably 95% or more, more preferably 98% or more.

After the aromatic ring-hydrogenation, part of the metal of the hydrogenation catalyst may be sometimes dissolved and the dissolved metal is desirably removed depending on applications. It is possible to remove or reduce the dissolved metal by passing the product through a zeta potential filter, an ion-exchange resin, or the like. The amount of the metal contained in thus obtained hydrogenated trimellitic acid is usually 1,000 ppm or less, preferably 100 ppm or less, more preferably 10 ppm or less.

In the case where part or all of 1,2-dicarboxylic anhydride ring part is opened into 1,2-dicarboxylic acid in the product after the aromatic ring-hydrogenation reaction of trimellitic acid, the 1,2-dicarboxylic acid part may be converted into an acid anhydride ring by subjecting the product to a heating treatment under reduced pressure.

With regard to the temperature employed on that occasion, the lower limit is 50° C. or higher, preferably 120° C. or higher and the upper limit is 250° C. or lower, preferably 200° C. or lower.

With regard to the degree of reduced pressure employed on that occasion, the lower limit is not particularly limited and the upper limit is 0.1 MPa, preferably 0.05 MPa.

As a method of converting the 1,2-dicarboxylic acid part into an acid anhydride ring, a method of treatment with an acid anhydride of an organic acid can be also employed in addition to the above-mentioned method of heating under reduced pressure. As the acid anhydride of an organic acid to be used on that occasion, there may be mentioned acetic anhydride, propionic anhydride, maleic anhydride, phthalic anhydride, and the like but acetic anhydride is suitably used in view of easiness of removal at the time when used excessively.

With regard to the temperature employed on that occasion, the lower limit is 30° C. or higher, preferably 50° C. or higher and the upper limit is 200° C. or lower, preferably 150° C. or lower.

The ratio of the compound having an acid anhydride ring thus obtained is usually 95% by mol, preferably 98% by mol, more preferably 99% by mol or more.

Next, a diester is synthesized from the thus obtained aromatic ring-hydrogenated trimellitic anhydride and a diol. As the esterification reaction (reaction of the carboxyl groups on the 4-positions of two molecules of the aromatic ring-hydrogenated trimellitic acid with a diol) on that occasion, a reaction usually known as an esterification reaction in organic synthesis can be arbitrarily employed. For example, there may be mentioned a method of esterification through direct dehydration from a carboxylic acid and an alcohol, a method of dehydrative condensation using a dehydrating reagent such as dicyclohexylcarbodiimide (abbreviated as DCC) and a combination of diethyl azodicarboxylate/triphenylphosphine, a method of an ester exchange reaction from a carboxylic acid and an alcohol ester of a carboxylic acid, a method of converting a carboxylic acid into an acid halide and subsequently reacting it with an alcohol in the presence of a basic substance, a method of producing a alicyclic tetracarboxylic acid by an ester exchange method (J. Polym. Sci. Part A, 4, 1531-1541 (1966)), and the like.

Among the aforementioned methods, a method of direct dehydration, a method of ester exchange, and a method of conversion into an acid halide are preferred in view of economical efficiency and reactivity.

The following specifically describes the method of conversion into an acid halide as one example but the method of producing the alicyclic tetracarboxylic anhydride having an ester group or a class of tetracarboxylic acid thereof of the invention is not particularly limited thereto. Moreover, as an example of conversion into an acid anhydride, the following describes a method of conversion of the aromatic ring-hydrogenated trimellitic anhydride into an acid chloride and producing a diester of the aromatic ring-hydrogenated trimellitic anhydride from it and a diol but a method of conversion into an acid bromide or an acid iodide other than the acid chloride can be entirely similarly employed.

In this method, an aromatic ring-hydrogenated trimellitic anhydride chloride is first synthesized. As a method of synthesis thereof, a usual method of synthesizing a carboxylic acid into a corresponding acid chloride can be used. Specific examples include a method of using thionyl chloride, a method of using oxalyl chloride, a method of using phosphorus trichloride, a method of using other acid chloride such as benzoyl chloride, and the like. Of these, use of thionyl chloride is preferred in view of easiness of removal of the chlorinating reagent used excessively by distillation.

As the method of producing the aromatic ring-hydrogenated trimellitic anhydride chloride using thionyl chloride, the method disclosed in JP-A-2004-203792 is known, for example.

Moreover, when the aromatic ring-hydrogenated trimellitic anhydride is chlorinated using a chlorinating agent, a catalyst such as N,N-dimethylformamide or pyridine can be also used but the reaction proceeds with no large trouble using no such a catalyst. Since the resulting chlorinated product is rather remarkably colored in some cases, care should be taken on coloration of the product in the case of an application where transparency of the polyesterimide film is of importance. In that case, it is preferred to produce it using no such a catalyst.

With regard to the amount of the chlorinating agent to be used, an equivalent amount for the reaction or an excess amount thereof is employed but the lower limit is usually 1 molar equivalent or more, preferably 5 molar equivalents or more, more preferably 10 molar equivalents or more. On the other hand, the upper limit is not particularly limited but is 100 molar equivalents or less, preferably 50 molar equivalents or more from the economical viewpoint.

The reaction may be carried out at room temperature but is usually carried out under heating. With regard to the temperature to be employed, the lower limit is 30° C., preferably 50° C. and the upper limit is a reflux temperature of the chlorinating agent to be used.

After the reaction, the chlorinating agent excessively used is removed. A method of removing the same is not particularly limited and distillation, extraction, and the like can be applied. In the case where it is removed by distillation, a solvent forming an azeotropic composition with the chlorinating agent may be added prior to the removal by distillation in order to improve efficiency. For example, in the case of removing thionyl chloride by distillation, it is possible to perform azeotropic distillation with adding benzene or toluene.

Purity of the resulting acid chloride can be further increased by recrystallization using a non-polar solvent as hexane or cyclohexane. However, since the acid chloride having a sufficiently high purity is usually obtained without such a purification operation, it may be used as it is in the next step depending on the situation.

Moreover, as a method of producing the aromatic ring-hydrogenated trimellitic anhydride chloride, it is also possible to treat 1,2,4-cyclohexanetricarboxylic acid directly with a chlorinating agent to achieve acid chloride formation and acid anhydride formation simultaneously in addition to the method of once converting 1,2-dicarboxylic acid part of 1,2,4-cyclohexanetricarboxylic acid obtained through nuclei-hydrogenation of trimellitic acid and subsequently converting the remaining carboxylic acid into acid chloride. On that occasion, the above-mentioned reaction conditions can be applied except that the amount of the chlorinating agent to be used is changed. With regard to the amount of the chlorinating agent to be used, the lower limit is usually 2 molar equivalents or more, preferably 5 molar equivalents or more, more preferably 10 molar equivalents or more. On the other hand, the upper limit is not particularly limited but is 100 molar equivalents or less, preferably 50 molar equivalents or less from the economical viewpoint.

At the time when the aromatic ring-hydrogenated trimellitic anhydride or 1,2,4-cyclohexanetricarboxylic acid is treated with a chlorinating agent to produce the aromatic ring-hydrogenated trimellitic anhydride chloride, the reaction may be carried out using a solvent. With regard to the solvent usable at that time, any solvent can be used without limitation so far as it is a solvent in which the chlorinating agent to be used and the aromatic ring-hydrogenated trimellitic anhydride chloride as a product are dissolved and which does not react with the chlorinating agent. Examples of the usable solvent include aromatic hydrocarbon solvents such as toluene and xylene, aliphatic hydrocarbon solvents such as hexane and heptane, ethereal solvents such as diethyl ether, tetrahydrofuran, monoethylene glycol dimethyl ether, and diethylene glycol dimethyl ether, ketone-based solvents such as acetone, methyl ethyl ketone, and methyl isobutyl ketone, ester-based solvents such as butyl acetate and γ-butyrolactone, amide-based solvents such as dimethylformamide, dimethylacetamide, and N-methylpyrrolidone, and the like. Of these, toluene, heptane, and tetrahydrofuran are preferred in view of solubility and stability. These solvents may be used singly or may be used as a mixture of any two or more solvents. With regard to the amount of the solvent to be used, the lower limit is usually 5% by weight, preferably 10% by weight and the upper limit is 50% by weight, preferably 40% by weight as a weight concentration of the aromatic ring-hydrogenated trimellitic anhydride or 1,2,4-cyclohexanetricarboxylic acid as a substrate.

Purity of the aromatic ring-hydrogenated trimellitic anhydride chloride thus obtained by purification as needed is usually 90% or more, preferably 95% or more, more preferably 98% or more. Main impurities include diacid chloride and triacid chloride (including stereoisomers) formed by acid chloride formation of a plurality of carboxyl groups of tricarboxylic acid resulting from ring opening of acid anhydride ring, decomposition products of dimethylformamide in the case of using dimethylformamide as a catalyst, dimethylamide of the aromatic ring-hydrogenated trimellitic acid, and the like. The amount of them present is preferably small and is usually 5% by mol or less, further preferably 3% by weight or less, more preferably 1% by weight or less.

Next, in the invention, the aromatic ring-hydrogenated trimellitic anhydride chloride thus obtained is esterified through reaction with a diol to synthesize a diester represented by the general formula (1). In this case, it is possible as a reaction to react it with not a diol but a diamine to form a diamide, followed by polyimidation of the resulting diacid anhydride as a starting material but there arise problems of high water absorbability and low toughness when finally converted into a resin, so that use of a diol is preferred.

A method of adding the reagents in the reaction of a diol with the acid chloride is not particularly limited and any addition method can be employed. For example, there can be employed a method of dissolving the diol and a basic substance in a solvent and slowly adding dropwise thereto the above aromatic ring-hydrogenated trimellitic anhydride chloride dissolved in a solvent or inversely a method of adding a mixed solution of the diol and the basic substance dropwise into the above aromatic ring-hydrogenated trimellitic anhydride chloride, a method of adding the basic substance dropwise into a mixed solution of the aromatic ring-hydrogenated trimellitic anhydride chloride and the diol, further a method of simultaneously adding a solution of the aromatic ring-hydrogenated trimellitic anhydride chloride and a solution of the basic substance into a solution of the diol, and the like.

In the reaction of the diol with the acid chloride in the presence of the basic substance, a white precipitate forms as the reaction proceeds. After filtration thereof, the precipitate was thoroughly washed with water to remove a hydrochloride formed through neutralization of the basic substance and the precipitate of the diester was dried under vacuum at high temperature to obtain a crude product of the objective alicyclic tetracarboxylic anhydride containing an ester group in high yields. Further recrystallization in an appropriate solvent according to necessity affords the alicyclic tetracarboxylic anhydride containing an ester group having an increased purity.

The diol usable at the synthesis of the alicyclic tetracarboxylic anhydride containing an ester group is not particularly limited but there may be usually used one having two hydroxyl groups in a monocyclic aromatic ring, one having two hydroxyl groups in an alicyclic skeleton, one having one hydroxyl group in each of both nuclei of a biphenyl skeleton, one having a structure where two phenols or alicyclic alcohols are bonded through a functional group such as a methylene group (—CH₂—), an ether group (—O—), an ester group (—C(O)O—), a keto group (—C(O)—), a sulfonyl group (—SO₂—), a sulfinyl group (—SO—), a sulfenyl group (—S—), or a 9,9-fluorenylidene group, one having two hydroxyl groups in a naphthalene skeleton, and one having two hydroxyl groups in a linear chain skeleton. As specific examples, examples of one having two hydroxyl groups in a monocyclic aromatic ring include hydroquinone, 2-methylhydroquinone, resorcinol, catechol, 2-phenylhydroquinone, and the like, examples of one having one hydroxyl group in each of both nuclei of a biphenyl skeleton include 4,4′-biphenol, 3,4′-biphenol, 2,2′-biphenol, 3,3′5,5′-tetramethyl-4,4′-biphenol, and the like, examples of one having a structure where two phenols or alicyclic alcohols are bonded through a divalent functional group include 4,4′-diphenyl ether, 4,4′-diphenyl sulfone, 4,4′-(9-fluorenylidene)diphenol, and the like, examples of one having two hydroxyl groups in a naphthalene skeleton include 2,6-naphthalenediol, 1,4-naphthalenediol, 1,5-naphthalenediol, 1,8-naphthalenediol, and the like, examples of one having two hydroxyl groups in an alicyclic skeleton include 1,4-dihydroxycyclohexane, 1,3-dihydroxycyclohexane, 1,2-dihydroxycyclohexane, 1,3-adamantanediol, dicyclopentadiene dihydrate, and the like, examples of one having two hydroxyl groups in a linear chain skeleton include ethylene glycol, propylene glycol, and the like, and examples of the other diol include cyclohexanedimethanol and the like. Of these, more preferred are diols having a cyclic skeleton, and furthermore, in view of required properties as polymers, hydroquinone, 4,4′-biphenol, 3,3′,5,5′-tetramethyl-4,4′-biphenol, 4,4′-(9-fluorenylidene)diphenol, 4,4′-methylenebisphenol, 4,4′-isopropylidenebisphenol (bisphenol A), 2,6-naphthalenediol, 1,4-dihydroxycyclohexane are particularly preferred. Moreover, two or more kinds of these diols can be used in combination.

With regard to the amount of the diol to be used, the upper limit is usually 0.6 equivalent, preferably 0.5 equivalent to the aromatic ring-hydrogenated trimellitic anhydride chloride. When the diol is used in an amount larger than the above amount, a half ester wherein only one of the diol is esterified is formed in a large amount, so that the case is not preferred. Moreover, the lower limit to be used is 0.3 equivalent, preferably 0.45 equivalent thereof. When the diol is used in an amount smaller than the above amount, the aromatic ring-hydrogenated trimellitic anhydride chloride remains in the system, so that the case is not preferred. Usually, 0.5 equivalent thereof is used.

The solvent usable at the synthesis of the alicyclic tetracarboxylic anhydride containing an ester group by reacting the aromatic ring-hydrogenated trimellitic anhydride chloride with the diol is not particularly limited and there may be mentioned ethereal solvents such as tetrahydrofuran, 1,4-dioxane, and 1,2-dimethoxyethane-bis(2-methoxyethyl)ether, aromatic amine solvents such as picoline, and piperidine, ketone-based solvents such as acetone and methyl ethyl ketone, aromatic hydrocarbon solvents such as toluene and xylene, halogen-containing solvents such as dichloromethane, chloroform, and 1,2-dichloroethane, amide-based solvents such as N-methyl-2-pyrrolidone, N,N-dimethylacetamide, N,N-diethylacetamide, and N,N-dimethylformamide, phosphorus-containing solvents such as hexamethylphosphoramide, sulfur-containing solvents such as dimethyl sulfoxide, ester-based solvents such as γ-butyrolactone, ethyl acetate and butyl acetate, nitrogen-containing solvents such as 1,3-dimethyl-2-imidazolidinone, aromatic solvents containing a hydroxyl group such as phenol, o-cresol, m-cresol, p-cresol, o-chlorophenol, m-chlorophenol, and p-chlorophenol, and the like. These solvents may be used singly or as a mixture of two or more thereof.

With regard to the concentration of the solute in the reaction of obtaining the alicyclic tetracarboxylic anhydride containing an ester group, the lower limit is 5% by weight, preferably 10% by weight, and the upper limit is 50% by weight, preferably 40% by weight. In consideration of the control of side reactions and the filtration step of precipitates, the reaction is more preferably carried out in the range of 10% by weight or more and 40% by weight or less.

At the synthesis of the alicyclic tetracarboxylic anhydride containing an ester group according to the invention, with regard to the reaction temperature to be employed, the lower limit is −10° C., preferably −5° C., more preferably 0° C. and the upper limit is 80° C., preferably 50° C., more preferably 20° C. When the reaction temperature is higher than 80° C., side reaction(s) might partially occur and thus yields might decrease, so that the case is not preferred.

Moreover, with regard to the reaction time to be employed, the lower limit is usually 5 minutes, preferably 10 minutes and the upper limit is not particularly limited and is usually 100 hours, preferably 24 hours.

The reaction is usually carried out under normal pressure but, if necessary, can be also carried out under elevated pressure or under reduced pressure. Usually, the reaction is carried out under nitrogen as a reaction atmosphere.

The reaction vessel may be either a tightly closed reaction vessel or an open reaction vessel but, in order to maintain the reaction system inert atmosphere, a vessel capable of being sealed with an inert gas is used in the case of an open one.

The basic substance is used for the purpose of neutralizing hydrogen chloride generated as the reaction proceeds. The kind of the basic substance to be used on this occasion is not particularly limited and organic tertiary amines such as pyridine, triethylamine, and N,N-dimethylaniline and inorganic basic substances such as potassium carbonate and sodium hydroxide can be used. Pyridine and triethylamine are preferred in view of availability in low costs and in view of easiness of reaction operations since they are liquid and rich in solubility. In addition, inorganic basic substances are preferred owing to availability in low costs.

With regard to the amount of the basic substance to be used, the lower limit is usually 1.0 molar equivalent, preferably 1.5 molar equivalents, more preferably 2.0 molar equivalents or more to the aromatic ring-hydrogenated trimellitic anhydride chloride. The upper limit is not particularly limited but is usually 30 molar equivalents, preferably 20 molar equivalents, more preferably 10 molar equivalents since the substance may contaminate the product and load for purification may increase when an excessive amount is used. When the amount of the basic substance is too large, load for purification of the objective product increases, so that the case is not preferred.

### <Purification Method of Alicyclic Tetracarboxylic Anhydride Containing an Ester Group or Class of Tetracarboxylic Acid Thereof>

For example, the reaction product obtained from the reaction of the aromatic ring-hydrogenated trimellitic anhydride chloride with the diol is a mixture of the objective product and a hydrochloride. In order to separate and remove the hydrochloride from the mixture, it is also possible to use a method of extracting and dissolving the precipitate with chloroform, ethyl acetate, or the like and washing the organic layer with water using a separating funnel but the hydrochloride can be completely removed by merely washing the precipitate thoroughly with water. The removal of the hydrochloride can be easily judged by analyzing the presence or absence of formation of white precipitate of silver chloride in a washing liquid with a 1% silver nitrate aqueous solution. On this occasion, the remaining amount of the chloride element is usually 1% by weight or less, preferably 0.1% by weight or less, more preferably 0.05% by weight or less.

At the operation of washing with water, the alicyclic tetracarboxylic anhydride containing an ester group is partially changed into an alicyclic tetracarboxylic acid containing an ester group though hydrolysis. However, the alicyclic tetracarboxylic acid containing an ester group formed through partial hydrolysis can be easily converted into the alicyclic tetracarboxylic anhydride containing an ester group by heating under reduced pressure.

With regard to the temperature employed on that occasion, the lower limit is 50° C., preferably 120° C. and the upper limit is 250° C., preferably 200° C.

With regard to the degree of reduced pressure employed on that occasion, the lower limit is not limited and the upper limit is 0.1 MPa, preferably 0.05 MPa.

With regard to the heating time employed on that occasion, the lower limit is usually 5 minutes, preferably 10 minutes and the upper limit is not particularly limited but is usually 100 hours, preferably 50 hours.

Moreover, as a method of ring re-closure in the case where the alicyclic tetracarboxylic acid containing an ester group is formed through hydrolysis, a method of treating the acid with an acid anhydride of an organic acid can be also employed in addition to the above-mentioned method of heating under reduced pressure. As the acid anhydride of an organic acid to be used on that occasion, there may be mentioned acetic anhydride, propionic anhydride, phthalic anhydride, and the like but acetic anhydride is preferably used in view of easiness of removal when used excessively.

With regard to the employed treating time with the acid anhydride of an organic acid, the lower limit is usually 5 minutes, preferably 10 minutes and the upper limit is not particularly limited but is usually 100 hours, preferably 24 hours.

With regard to the treating temperature employed on that occasion, the lower limit is 0° C., preferably 20° C., more preferably 50° C. and the upper limit is 250° C., preferably 200° C., more preferably 150° C.

On that occasion, a solvent may be used as needed. The solvent to be used on that occasion is not particularly limited but there may be preferably used aromatic hydrocarbon solvents such as toluene and xylene, aliphatic hydrocarbon solvents such as hexane and heptane, ethereal solvents such as diethyl ether, tetrahydrofuran, monoethylene glycol dimethyl ether, and diethylene glycol dimethyl ether, ketone-based solvents such as acetone, methyl ethyl ketone, and methyl isobutyl ketone, ester-based solvents such as ethyl acetate, butyl acetate and γ-butyrolactone, amide-based solvents such as dimethylformamide, dimethylacetamide, and N-methylpyrrolidone, carboxylic acid solvents such as acetic acid, formic acid, and propionic acid, and the like. These solvents may be used singly or may be used as a mixture of any two or more solvents.

It is also possible to further purify the thus obtained alicyclic tetracarboxylic anhydride containing an ester group. As a purification method in that case, any of recrystallization, sublimation, washing, treatment with active carbon, column chromatography, and the like can be arbitrarily performed. In addition, it is possible to repeat the purification method or to perform a combination thereof.

The purity of the thus obtained alicyclic tetracarboxylic anhydride containing an ester group of the invention is usually 90% or more, preferably 95% or more, more preferably 98% or more as an area ratio of peaks obtained, for example, on analysis such as high performance liquid chromatography with a differential refractometry detector.

The substances contained as impurities include monoester compound wherein only one hydroxyl group of the diol is esterified, a ring-closing agent when an acid anhydride such as acetic anhydride is used as the ring closing agent at purification, and the like. Since these impurities contains one acid anhydride structure in the molecule, they function as polymerization-terminating agents at the polymerization with an diamine, so that it is necessary to remove them from the alicyclic tetracarboxylic anhydride containing an ester group as far as possible. The content of the monoacid anhydride such as acetic anhydride contained in the alicyclic tetracarboxylic anhydride containing an ester group is preferably 10% by mol or less, more preferably 5% by mol or less, further preferably 2% by mol or less. When the monoacid anhydride is present in an amount larger than the content, there is a possibility that the polymerization degree is not increased at the polymerization with a diamine.

Moreover, the yield of the alicyclic tetracarboxylic anhydride containing an ester group of the invention synthesized by esterification of the above hydrogenated trimellitic acid and the diol is usually 10% by mol or more, preferably 20% by mol or more, further preferably 30% by mol or more, more preferably 50% by mol or more after purification.

### <Storage Method of Alicyclic Tetracarboxylic Anhydride Containing an Ester Group or Class of Tetracarboxylic Acid Thereof>

With regard to the storage of the alicyclic tetracarboxylic anhydride containing an ester group, it is desirable to store it at low temperature with avoiding high humidity in order to prevent ring opening of the acid anhydride ring by hydrolysis. Specifically, when stored in a well-sealed vessel in a refrigerator, it can be stored for a long time. Moreover, with regard to the alicyclic tetracarboxylic anhydride containing an ester group, in order to prevent moisture absorption, it can be used in the next polymerization reaction immediately after purification. The storage period on that occasion is usually 100 hours or less, preferably 50 hours or less, more preferably 24 hours or less.

The alicyclic tetracarboxylic acid containing an ester group can be stored at room temperature for a long time without requiring particular regulation of humidity.

### <Process for Producing Alicyclic Polyesterimide Precursor>

A process for producing alicyclic polyesterimide precursor of the invention is not particularly limited and any known processes can be applied. Usually, the alicyclic polyesterimide precursor can be easily produced by reacting substantially equimolar amount of a class of diamine and the alicyclic tetracarboxylic dianhydride containing an ester group or a class of tetracarboxylic acid thereof in a polymerization solvent. On this occasion, it is preferred to use a compound represented by the above formula (1) as the alicyclic tetracarboxylic dianhydride containing an ester group.

Moreover, it is also possible to use a compound represented any of the following formulae (6) to (8) derived from the above formula (1) as a class of alicyclic tetracarboxylic acid containing an ester group.

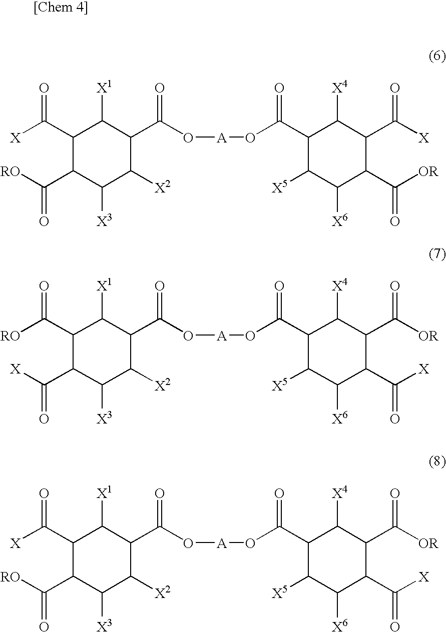

In the formulae (6) to (8), R is an alkyl group having 1 to 12 carbon atoms and X is a hydroxyl group or a halogen atom (any of fluorine, chlorine, bromine, and iodine). Moreover, the structure of A is not particularly limited so far as A can be bonded to the carboxyl groups at the two sites so as to form the above structure. Specifically, in the formulae (4) to (6), A can be any divalent group and is preferably a divalent group containing an aromatic group or an aliphatic group. Furthermore, A may be a structure wherein a plurality of aromatic group(s) and/or aliphatic group(s) are bonded one another through a functional group such as a methylene group (—CH₂—), an ether group (—O—), an ester group (—C(O)O—), a keto group (—C(O)—), a sulfonyl group (—SO₂—), a sulfinyl group (—SO—), a sulfenyl group (—S—), a 9,9-fluorenylidene group, or the like. Of these, when A is a structure containing at least one aromatic or aliphatic cyclic structure, thermal resistance increases when converted into a resin, so that the case is more preferred. Further preferably, there may be mentioned a phenylene group, a naphthylene group, a cyclohexylene group, a biphenylene group, a diphenyl ether group, a diphenyl sulfone group, a methylenediphenyl group, an isopropylidenediphenyl group, a 4,4′-(9-fluorenylidene)diphenyl group, a dicyclohexylether group, a linear aliphatic group, and the like, which are each a divalent group. Of these, a phenylene group, a biphenylene group, a biphenyl ether group, a biphenyl sulfone group, and the like are particularly preferred in view of their rigid structure.

Moreover, X¹, X², X³, X⁴, X⁵, and X⁶ in the above formulae (6) to (8) each independently represents a hydrogen atom, a halogen atom, a nitrile group, a nitro group, an alkyl group, an alkenyl group, an alkynyl group, an alkoxy group, an amino group, or an amide group. The carbon number of the alkyl group, alkenyl group, alkynyl group, alkoxy group, amino group, or amide group is preferably from 1 to 10. More specifically, examples of the alkyl group include a methyl group, an ethyl group, an n-propyl group, an i-propyl group, an n-butyl group, and the like. Examples of the alkoxy group include a methoxy group, an ethoxy group, an n-propoxy group, an i-propoxy group, an n-butoxy group, and the like. Of these, a hydrogen atom or a halogen atom is preferred in view of easy availability of the starting material.

Preferred structures as combinations of A and X¹, X², X³, X⁴, X⁵, and X⁶ are those wherein A is a group having a cyclic structure and X¹, X², X³, X⁴, X⁵, and X⁶ each is independently composed of a halogen atom or a hydrogen atom. More preferred is one wherein A is a group having a cyclic structure and all of X¹, X², X³, X⁴, X⁵, and X⁶ are composed of hydrogen atoms.

The compounds of the formulae (6) to (8) can be synthesized as dicarboxylic acid dialkyl esters by reacting the compound of the formula (1) with an alcohol dried beforehand to ring-open the acid anhydride ring (X═OH). On this occasion, the product is usually obtained as a mixture of the compounds represented by the formulae (6) to (8). Furthermore, when the carboxyl moiety formed by opening the acid anhydride ring is chlorinated with a chlorinating agent such as thionyl chloride, an acid chloride can be synthesized (X═Cl). For the polymerization of the alicyclic polyesterimide precursor of the invention, the mixture of the compounds (6) to (8) can be used but each isolated compound therefrom may be used. Moreover, the use of the mixture does not affect the physical properties after imidation.

The diamine to be used for production of the alicyclic polyesterimide precursor according to the invention can be freely selected within a range which does not remarkably impair the required properties of the alicyclic polyesterimide. Specific examples of the diamine usable include, as aromatic diamines, 3,5-diaminobenzotrifluoride, 2,5-diaminobenzotrifluoride, 3,3′-bistrifluoromethyl-4,4′-diaminobiphenyl, 3,3′-bistrifluoromethyl-5,5′-diaminobiphenyl, bis(trifluoromethyl)-4,4′-diaminodiphenyl, bis(fluorinated alkyl)-4,4′-diaminodiphenyl, dichloro-4,4′-diaminodiphenyl, dibromo-4,4′-diaminodiphenyl, bis(fluorinated alkoxy)-4,4′-diaminodiphenyl, diphenyl-,4′-diaminodiphenyl, 4,4′bis(4-aminotetrafluorophenoxy)tetrafluorobenzene, 4,4′-bis(4-aminotetrafluorophenoxy)octafluorobiphenyl, 4,4′-binaphthylamine, o-, m-, p-phenylenediamine, 2,4-diaminotoluene, 2,5-diaminotoluene, 2,4-diaminoxylene, 2,4-diaminodurene, dimethyl-4,4′-diaminodiphenyl, dialkyl-4,4′-diaminodiphenyl, dimethoxy-4,4′-diaminodiphenyl, diethoxy-4,4′-diaminodiphenyl, 4,4′-diaminodiphenylmethane, 4,4′-diaminodiphenyl ether, 3,4′-diaminodiphenyl ether, 4,4′-diaminodiphenyl sulfone, 3,3′-diaminodiphenyl sulfone, 4,4′-diaminobenzophenone, 3,3′-diaminobenzophenone, 1,3-bis(3-aminophenoxy)benzene, 1,3-bis(4-aminophenoxy)benzene, 1,4-bis(4-aminophenoxy)benzene, 4,4′-bis(4-aminophenoxy)biphenyl, bis(4(3-aminophenoxy)phenyl) sulfone, bis(4-(4-aminophenoxy)phenyl) sulfone, 2,2-bis(4-(4-aminophenoxy)phenyl)propane, 2,2-bis(4-(4-aminophenoxy)phenyl)hexafluoropropane, 2,2-bis(4-(3-aminophenoxydi)phenyl)propane, 2,2-bis(4-(3-aminophenoxy)phenyl)hexafluoropropane, 2,2-bis(4-(4-amino-2-trifluoromethylphenoxy)phenyl)hexafluoropropane, 2,2-bis(4-(3-amino-5-trifluoromethylphenoxy)phenyl)hexafluoropropane, 2,2-bis(4-aminophenoxy)hexafluoropropane, 2,2-bis(3-aminophenoxy)hexafluoropropane, 2,2-bis(3-amino-4-hydroxyphenyl)hexafluoropropane, 2,2-bis(3-amino-4-methylphenyl)hexafluoropropane, 4,4′-bis(4-aminophenoxy)octafluorobiphenyl, 4,4′-diamnobenzanilide, and the like and two or more thereof can be used in combination.

Examples as aliphatic diamines include 4,4′-methylenebis(cyclohexylamine), isophorondiamine, trans-1,4-diaminocyclohexane, cis-1,4-diaminocyclohexane, 1,4-cyclohexanebis(methylamine), 2,5-bis(aminomethyl)bicyclo[2.2.1]heptane, 2,6-bis(aminomethyl)bicyclo[2.2.1]heptane, 3,8-bis(aminomethyl)tricyclo[5.2.1.0]decane, 1,3-diaminoadamantane, 2,2-bis(4-aminocyclohexyl)propane, 2,2-bis(4-aminocyclohexyl)hexafluoropropane, 1,3-propanediamine, 1,4-tetramethylenediamine, 1,5-pentamethylenediamine, 1,6-hexamethylenediamine, 1,7-heptamethylenediamine, 1,8-octamethylenediamine, 1,9-nonamethylenediamine, and the like. Moreover, two or more thereof can be used in combination.

Furthermore, diamines containing a siloxane group, such as 1,3-bis(3-aminopropyl)-1,1,3,3-tetramethyldisiloxane can be also used.

Of these diamines, as the aromatic diamines, monocyclic phenylenediamine compounds such as o-, m-, and p-phenylenediamines and diaminodiphenyl compounds such as 4,4′-diaminodiphenyl, 4,4′-diaminodiphenyl sulfone, 4,4′-diaminodiphenylmethane, and 4,4′-diaminodiphenyl ether are preferred. Of these, owing to easy availability and good physical properties of the resins obtained, p-phenylenediamine, 4,4′-diaminodiphenyl ether, and 4,4′-diaminodiphenyl are more preferred. As the aliphatic diamines, alicyclic diamines such as 4,4′-methylenebis(cyclohexylamine), trans-1,4-diaminocyclohexane, and isophorondiamine are more preferred owing to ring structure and easy availability. Furthermore, trans-1,4-diaminocyclohexane is more preferred owing to good physical properties of the resins obtained.

Purification may be performed prior to the use of these diamines. As purification methods, any of recrystallization, sublimation, washing, treatment with active carbon, column chromatography, and the like can be arbitrarily performed. In addition, it is possible to repeat the purification method or to perform a combination thereof.

These diamines are preferably high purity since polymerization reactivity increases. The purity of the diamine to be usually used is 95% or more, preferably 97% or more, more preferably 99% or more.

The alicyclic polyesterimide precursor can be formed by polymerization from the tetracarboxylic dianhydride of the formula (1) and substantially equimolar amount of a diamine. More specifically, the precursor can be obtained by the following method.

The reaction is carried out by mixing the diamine and the tetracarboxylic dianhydride of the formula (1) in the presence of a solvent.

On this occasion, the ratio of the tetracarboxylic dianhydride and the diamine to be used is preferably 1:0.8 to 1.2 as a molar ratio. Similarly to the usual polycondensation reaction, the molecular weight of the resulting polyamidic acid increases as the molar ratio becomes close to 1:1.

A method of charging these diamine and acid anhydride into a reaction vessel can be arbitrarily selected. For example, a method of dissolving the diamine in a solvent and gradually adding powder of the tetracarboxylic dianhydride of the formula (1) thereto, inversely a method of gradually adding the diamine to the solution of the tetracarboxylic dianhydride, and further, a method of simultaneously adding the diamine and the powder of the tetracarboxylic dianhydride to a reaction vessel into which a solvent is charged beforehand. Of these, the method of dissolving the diamine in a solvent and gradually adding powder of the tetracarboxylic dianhydride is advantageously employed based on the solubility of the reagents to a solvent.

With regard to the reaction temperature, when it is too low, the solubility of the reagents reduces and a sufficient reaction rate is not obtained and when it is too high, the proceeding of the reaction becomes difficult to control. Therefore, the cases are not preferred. The lower limit is −20° C., preferably −10° C., more preferably 0° C. and the upper limit is 150° C., preferably 100° C., more preferably 60° C.

The reaction time can be determined without particular limitation but, in order to achieve a sufficient conversion rate of the reagents, the lower limit is 10 minutes, preferably 30 minutes, more preferably 1 hour and the upper limit is not particularly limited but it is not necessary to extend the reaction time beyond a required time so far as the reaction is completed. For example, 100 hours, preferably 50 hours, or more preferably 30 hours is employed.

The polymerization reaction is carried out using a solvent. The solvent to be used on this occasion is structurally not particularly limited so far as the diamine and the tetracarboxylic acid of the invention as starting monomers do not react with the solvent and these starting materials are dissolved in the solvent. As specific examples, there may be preferably employed amide solvents such as N,N-dimethylformamide, N,N-dimethylacetamide, and N-methylpyrrolidone, cyclic ester solvents such as γ-butyrolactone, γ-valerolactone, δ-valerolactone, γ-caprolactone, ∈-caprolactone, and α-methyl-γ-butyrolactone, carbonate solvents such as ethylene carbonate and propylene carbonate, lactam solvents such as caprolactam, ethereal solvents such as dioxane, glycol-based solvents such as triethylene glycol, phenol-based solvents such as m-cresol, p-cresol, 3-chlorophenol, 4-chlorophenol, 4-methoxyphenol, and 2,6-dimethylphenol, acetophenone, 1,3-dimethyl-2-imidazolidinone, sulfolane, dimethyl sulfoxide, tetramethylurea, and the like. Furthermore, the other general organic solvents, namely, phenol, o-cresol, butyl acetate, ethyl acetate, isobutyl acetate, propylene glycol methyl acetate, ethyl cellosolve, butyl cellosolve, 2-methyl cellosolve acetate, ethyl cellosolve acetate, butyl cellosolve acetate, tetrahydrofuran, dimethoxyethane, diethoxyethane, dibutyl ether, diethylene glycol dimethyl ether, methyl isobutyl ketone, diisobutyl ketone, cyclohexanone, methyl ethyl ketone, acetone, butanol, ethanol, xylene, toluene, chlorobenzene, terpene, mineral spirit, petroleum naphtha-based solvents, and the like can be used in combination with the above solvents. Of these, owing to high solubility of the starting materials, aprotic solvents such as N,N-dimethylformamide, N,N-dimethylacetamide, N-methyl-2-pyrrolidone, dimethyl sulfoxide, and γ-butyrolactone are preferred.

With regard to the amount of the solvent to be used, it is preferred to use a solvent in such an amount that the weight concentration of total amount of the tetracarboxylic dianhydride and the diamine as starting materials falls within the following range. Namely, the concentration is 0.1% by weight or more, preferably 1% by weight or more, more preferably 5% by weight or more and the upper limit is not particularly limited but, in view of solubility of the tetracarboxylic dianhydride, is 80% by weight or less, preferably 50% by weight or less, more preferably 30% by weight or less. By performing polymerization in this concentration range of the tetracarboxylic dianhydride, a homogeneous solution of a polyimide precursor having a high polymerization degree can be obtained. In order to impart film toughness to the objective polyesterimide, the polymerization degree is preferably as high as possible. When polymerization is performed at lower concentration than the above concentration range, a sufficient polymerization degree of the polyimide precursor might not be obtained and thus the finally obtained polyimide film might become brittle, so that the case is not preferred. In the case of using an alicyclic diamine as the diamine, it takes a long polymerization time to dissolve the formed salt at higher concentration until disappearance thereof and hence decrease in productivity might be invited.

If necessary, an inorganic salt may be used as a catalyst at the production of the precursor. Examples of the inorganic salt to be used on this occasion include alkaline metal halides such as LiCl, NaCl, and LiBr, alkaline earth metal halides such as CaCl₂, and metal halides such as ZnCl₂. Of these, metal halides such as LiCl, CaCl₂, and ZnCl₂ are particularly preferred.

The reaction is preferably carried out under stirring in the course thereof.

With regard to the weight-average molecular weight of the alicyclic polyesterimide precursor of the invention thus obtained, the lower limit is 3,000, preferably 5,000 and the upper limit is 150,000, preferably 100,000. The molecular weight can be measured by gel permeation chromatography (GPC) or the like, for example.

Moreover, the logarithmic viscosity of the obtained alicyclic polyesterimide precursor is not particularly limited but as preferable logarithmic viscosity, the lower limit is 0.3 dL/g, preferably 0.5 dL/g, more preferably 0.7 dL/g. On the other hand, the upper limit is 5.0 dL/g, preferably 3.0 dL/g, more preferably 2.0 dL/g. The logarithmic viscosity can be measured using Ostwald viscometer, for example.

It is possible to remove foreign particles contained by filtration of a solution of the alicyclic polyesterimide precursor. Removal of the foreign particles is important particularly in the case where the resin is utilized in optical uses. With regard to the amount of the foreign particles in the alicyclic polyesterimide precursor obtained in the invention, usually, insoluble fine particles having a projected area circle-corresponding diameter of 5 to 20 μm is 5,000 pieces or less, preferably 3,000 pieces or less, more preferably 1,000 pieces or less per 1 g of the precursor. The number of the foreign particles can be counted, for example, by a microscopic method wherein size and number of the insoluble fine particles are counted on a microscopic image. Specifically, they can be easily counted utilizing a particle size image processing apparatus such as XV-1000 manufactured by Keyence Corporation, for example.

Moreover, synthesis of the alicyclic polyesterimide precursor of the invention is possible by low-temperature solution polycondensation according to a known method from a diacid halide of the corresponding tetracarboxylic acid dialkyl ester and a diamine (for example, the method described in High Performance Polymers, 10, 11 (1988) and the like). Specifically, the synthesis is performed by reacting the diamine with the tetracarboxylic acid derivative represented by any of the formulae (6) to (8) (X is a halogen atom) in the presence of a solvent.

A method of charging these diamine and tetracarboxylic acid derivative represented by any of the formulae (6) to (8) into a reaction vessel can be arbitrarily selected. For example, it is possible to employ a method of dissolving the diamine in a solvent and gradually adding the tetracarboxylic acid derivative thereto, inversely a method of gradually adding the diamine to the solution of the tetracarboxylic acid derivative, and further, a method of simultaneously adding the diamine and the tetracarboxylic acid derivative to a reaction vessel into which a solvent has been charged beforehand. Of these, the method of dissolving the diamine in a solvent and gradually adding the tetracarboxylic acid derivative is advantageously employed owing to easiness of reaction control.

With regard to the reaction temperature, when it is too low, the solubility of the reagents reduces and a sufficient reaction rate is not obtained and when it is too high, the proceeding of the reaction becomes difficult to control. Therefore, the cases are not preferred. The lower limit is −20° C., preferably −10° C., more preferably 0° C. and the upper limit is 150° C., preferably 100° C., more preferably 80° C.

The reaction time can be determined without particular limitation but the lower limit is 10 minutes, preferably 30 minutes, more preferably 1 hour. The upper limit is not particularly limited but is 150 hours, preferably 100 hours, more preferably 50 hours.

The polymerization reaction is carried out using a solvent. As the solvent to be used on this occasion, the solvents to be used in the reaction of the diamine and the tetracarboxylic dianhydride described above can be used.

With regard to the amount of the solvent to be used, it is preferred to use a solvent in such an amount that the weight concentration of total amount of the tetracarboxylic acid derivative represented by any of the formulae (6) to (8) and the diamine as starting materials falls within the following range. The lower limit of the concentration is 0.1% by weight, preferably 1% by weight, more preferably 5% by weight and the upper limit is not particularly limited but is 80% by weight, preferably 50% by weight, more preferably 30% by weight in view of the solubility of the tetracarboxylic dianhydride.

At the reaction, a basic substance may be used. The basic substance usable in the invention is a tertiary amine or an inorganic basic substance. Specifically, aromatic tertiary amines such as pyridine, aliphatic tertiary amines such as triethylamine and N-methylpiperidine, and inorganic basic substances such as potassium carbonate, sodium carbonate, and sodium salt and sodium hydrogen salt of phosphoric acid can be used. Of these, pyridine and triethylamine are preferred in view of easy availability and operability. These basic substances are preferably added after dissolved in a solvent to be used at the reaction beforehand. The amount of the basic substance to be used can be arbitrarily changed depending on the amount of the acid contained in the tetracarboxylic acid derivatives represented by the formulae (6) to (8). Of course, it is possible to use no basic substance when any acid generated during the reaction is not present in the tetracarboxylic acid derivative. With regard to the amount of the basic substance in the case where an acid is generated, the lower limit is 2 molar equivalents, preferably 3 molar equivalents and the upper limit is 10 molar equivalents, preferably 5 molar equivalents to the number of mol of the tetracarboxylic acid derivative used in the polymerization.

The reaction is preferably carried out under stirring in the course of the reaction.

The polymerization reaction of the diamine with the tetracarboxylic acid derivatives represented by the formulae (6) to (8) can be also carried out through surface polycondensation. In the surface polycondensation, the solvent used is characteristic. Namely, the diamine is dissolved in an aqueous solution into which a basic substance such as a tertiary amine is dissolved. On the other hand, the tetracarboxylic acid derivatives represented by the formulae (6) to (8) (the case where X is a chlorine atom) is dissolved in a non-polar organic solvent which does not dissolve in water. As the non-polar solvent to be used on this occasion, aromatic solvents such as toluene and xylene and aliphatic hydrocarbon solvents such as cyclohexane, hexane, and heptane are used.

In the case where the polymerization reaction is carried out in the surface polycondensation, it is possible to obtain the polyesterimide precursor by mixing and stirring these two solutions vigorously. On this occasion, there arises no trouble even when the charged amounts of the diamine and the tetracarboxylic acid derivative are not equimolar.

Furthermore, the alicyclic polyesterimide precursor of the invention can be produced in the presence of a condensing agent using the tetracarboxylic acid derivatives represented by the formulae (6) to (8) (the case where X is a hydroxyl group) and an equimolar amount of the diamine. For example, using triphenyl phosphite equimolar to the diamine as a condensing agent, it is also possible to perform direct polycondensation in the presence of pyridine. Moreover, it is also possible to perform direct polycondensation also using N,N-dicyclohexylcarbodiimide as the other condensing agent.

Moreover, the production of the alicyclic polyesterimide precursor of the invention is also possible by low-temperature solution polycondensation of a disilyl compound of the diamine with the tetracarboxylic dianhydride of the formula (1) or the tetracarboxylic acid derivatives represented by the formulae (6) to (8) (the case where X is a chlorine atom) in the same manner as above according to a known method (Kobunshi Toronkai Yokoshu, 49, 1917 (2000)).

The alicyclic polyesterimide or precursor thereof contains at least one of the units of the above general formulae (4) to (5) which are characteristics of the invention. Specifically, at the time of obtaining the alicyclic polyesterimide of the invention, the other acid dianhydride or tetracarboxylic acid may be mixed in addition to the alicyclic tetracarboxylic anhydride having an ester group or a class of tetracarboxylic acid thereof of the invention and copolymerized. The acid dianhydride usable on that occasion is not particularly limited but examples thereof include aromatic acid dianhydride having one benzene ring, such as pyromellitic acid, aromatic acid dianhydride having two benzene rings, such as 3,3′,4,4′-biphenyltetracarboxylic dianhydride (BPDA), 2,3′,3,4′-biphenyltetracarboxylic dianhydride (a-BPDA), 3,3″,4,4′-diphenylsulfonetetracarboxylic dianhydride (DSDA), 3,3′,4,4′-benzophenonetetracarboxylic dianhydride (BTDA), 2,2′,3,3′-benzophenonetetracarboxylic dianhydride, 3,3′,4,4′-oxydiphthalic anhydride (ODPA), bis(2,3-dicarboxyphenyl)ether dianhydride (a-ODPA), bis(3,4-dicarboxyphenyl)ether dianhydride, bis(3,4-dicarboxyphenyl)methane dianhydride, 2,2′-bis(3,4-dicarboxyphenyl)propane dianhydride (BDCP), 2,2′-bis(2,3-dicarboxyphenyl)propane dianhydride, 2,2′-bis(3,4-dicarboxyphenyl)hexafluoropropane dianhydride (BDCF), 2,2′-bis(2,3-dicarboxyphenyl)hexafluoropropane dianhydride, aromatic acid dianhydride having a naphthalene skeleton, such as 2,3,6,7-naphthalenetetracarboxylic dianhydride, 1,2,5,6-naphthalenetetracarboxylic dianhydride, and 1,4,5,8-naphthalenetetracarboxylic dianhydride, and aromatic acid dianhydride having an anthracene skeleton, such as 2,3,6,7-anthracenecarboxylic dianhydride and 1,2,5,6-anthracenecarboxylic dianhydride.

On the other hand, examples of the alicyclic anhydride usable include linear aliphatic tetracarboxylic dianhydrides such as 1,2,3,4-butanetetracarboxylic dianhydride and ethylenetetracarboxylic dianhydride, tetracarboxylic dianhydrides having an alicyclic structure, such as 1,2,3,4-cyclobutanetetracarboxylic dianhydride, 1,3-dimethyl-1,2,3,4-cyclobutanetetracarboxylic dianhydride, 1,2,4,5-cyclopentanetetracarboxylic dianhydride, 1,2,3,4-cyclopentanetetracarboxylic dianhydride, 1,2,4,5-cyclohexanetetracarboxylic dianhydride, bicyclo[2.2.2]oct-7-ene-2,3,5,6-tetracarboxylic dianhydride, dicyclohexyl-3,4,3′,4′-tetracarboxylic dianhydride (BPDA hydrogenated product), 2,3,5-tricarboxycyclopentylacetic dianhydride, 3,4-dicarboxy-1,2,3,4-tetrahydro-1-naphthalenesuccinic dianhydride, and bicyclo[3,3,0]octane-2,4,6,8-tetracarboxylic dianhydride, and the like.

The ratio of these acid dianhydride and alicyclic tetracarboxylic dianhydride containing an ester group of the invention to be used can be arbitrarily determined depending on physical properties of the resin to be obtained but the amount of the alicyclic tetracarboxylic dianhydride containing an ester group of the invention to be used is preferably 5% by mol, more preferably 10% by mol.

If necessary, an alicyclic polyesterimide precursor in a solution state can be isolated. For example, the alicyclic polyesterimide precursor can be isolated as a powder by adding a solution of the alicyclic polyesterimide precursor to a poor solvent such as water, methanol, or acetone to precipitate the alicyclic polyesterimide precursor and removing the solvent by drying or the like from the solid obtained through filtration. In this connection, if necessary, the powder can be dissolved in the reaction solvent described above to form a solution again. By repeating the operations, the alicyclic polyesterimide precursor of the invention can be purified.

### <Process for Producing Alicyclic Polyesterimide>

As the process for synthesizing the alicyclic polyesterimide of the invention, there may be mentioned (i) a process of obtaining it from the alicyclic polyesterimide precursor and (ii) a process of obtaining it without intervening the alicyclic polyesterimide precursor. As (i) the process of obtaining it from the alicyclic polyesterimide precursor, a heating imidation and a chemical imidation are included. However, the process for producing the alicyclic polyesterimide of the invention is not limited to the following production process.

(i) Process of Obtaining it from the Alicyclic Polyesterimide Precursor

The alicyclic polyesterimide of the invention can be produced by cyclic imidation reaction of the alicyclic polyesterimide precursor obtained in the above process.

On this occasion, the producible form of the alicyclic polyesterimide is a film, a powder, a molded article, and a solution.

The film of the alicyclic polyesterimide can be produced, for example, in the following manner. First, a polymerization solution (varnish) of the alicyclic polyesterimide precursor is applied on a substrate such as glass, copper, aluminum, silicon, a quartz plate, a stainless plate, or a capton film by casting. As a method for the application, the alicyclic polyesterimide solution obtained as described above can be applied in a uniform height by adding the solution onto the above-described substrate dropwise and casting the solution by rubbing over a support whose height is fixed. On this occasion, it is possible to use a device such as a doctor blade. In addition, as the other method for the application, any methods such as a spin-coating method, a printing method, and an inkjet method can be employed without limitation so far as the method can apply the solution in a predetermined thickness.

At the application of the alicyclic polyesterimide precursor on the substrate, a solvent is used to adjust the viscosity to a suitable one for application. With regard to the viscosity on this occasion, the lower limit is 1 poise, preferably 5 poises, and the upper limit is 100 poises, preferably 80 poises.

Since the applied coated film contains the solvent, it is then dried. With regard to the temperature for drying to be employed on that occasion, the lower limit is usually 20° C., preferably 40° C., more preferably 60° C. On the other hand, the upper limit is 200° C., preferably 150° C., more preferably 100° C.

The time for drying is not particularly limited so far as the solvent is removed to some extent. The lower limit is 10 minutes, preferably 30 minutes, more preferably 1 hour and the upper limit is not particularly limited and is 50 hours, preferably 30 hours, and more preferably 10 hours.

Drying may be performed under reduced pressure. The degree of reduced pressure to be employed on that occasion is usually 0.05 MPa or less, preferably 0.01 MPa or less, more preferably 0.001 MPa or less.

Usually, the remaining amount of the solvent after drying is 70% by weight or less, preferably 50% by weight or less, more preferably 30% by weight or less.

The dried alicyclic polyesterimide precursor film thus obtained is imidated on the substrate by heating at high temperature under vacuum in an inert gas such as nitrogen or in the air. This method is referred to as heating imidation.

With regard to the temperature to be employed on this occasion, the lower limit is 180° C., preferably 200° C., more preferably 250° C. On the other hand, it is heated at 500° C., preferably 400° C., more preferably 350° C. as an upper limit. When the heating temperature is 180° C. or lower, the cyclization reaction of the cyclizing imidation reaction might be insufficient, so that the case is not preferred. Also, when the temperature is too high, there is a possibility of coloration of the formed alicyclic polyesterimide film, so that the case is not preferred. Moreover, the imidation is desirably performed under vacuum or in an inert gas but it may be performed in the air when the temperature for the imidation reaction is not too high. The degree of reduced pressure to be employed in the case where the heating imidation is performed under reduced pressure is usually 0.05 MPa or less, preferably 0.01 MPa or less, more preferably 0.001 MPa or less.

With regard to the time for heating, a time during which the cyclizing imidation sufficiently proceeds is employed. The lower limit is usually 5 minutes, preferably 10 minutes, more preferably 20 minutes and the upper limit is not particularly limited and is 20 hours, preferably 10 hours, and more preferably 5 hours.

Moreover, it is also possible to carry out the chemical imidation reaction by immersing the alicyclic polyesterimide precursor film in a solution containing a dehydrating reagent. The reaction is preferably carried out in the presence of a tertiary amine.

The tertiary amine usable on this occasion includes aromatic tertiary amines such as pyridine and aliphatic tertiary amines such as triethylamine and N-methylpiperidine. Of these, pyridine and triethylamine are preferred in view of easy availability and good reactivity.

With regard to the amount of the tertiary amine to be used, the lower limit is usually 0.1 molar equivalent, preferably 0.5 molar equivalent, more preferably 1.0 molar equivalent to the amide group and the upper limit is usually 30 molar equivalents, preferably 20 molar equivalents, more preferably 10 molar equivalents.

Moreover, as the dehydrating reagent usable, there may be mentioned acid anhydrides such as acetic anhydride, propionic anhydride, and trifluoromethanesulfonic anhydride and carbodiimides such as N,N-dicyclohexylcarbodiimide. Of these, acetic anhydride, trifluoromethanesulfonic anhydride, and carbodiimides such as N,N-dicyclohexylcarbodiimide are preferred and acetic anhydride is more preferred in view of easy availability and economical efficiency.

On that occasion, with regard to the amount of the dehydrating reagent to be used, the lower limit is usually 1.0 molar equivalent, preferably 2.0 molar equivalents, more preferably 4.0 molar equivalents and the upper limit is not particularly limited but is usually 50 molar equivalents, preferably 30 molar equivalents, more preferably 20 molar equivalents to the number of mol of the amidic acid contained in the alicyclic polyesterimide precursor. The treatment with the dehydrating reagent may be carried out at room temperature and the reagent may be used under heating in the case where the reaction proceeds slowly.

Thus, in the cyclizing imidation reaction, heating or the dehydrating reagent is preferably used but the reaction can be carried out in combination with heating and the dehydrating reagent.

Moreover, as another embodiment of the heating imidation, the solution (varnish) of the alicyclic polyesterimide of the invention can be easily produced by heating the polymerization solution of the alicyclic polyesterimide precursor as it is or in a solution after appropriate dilution thereof with the same solvent.

The concentration of the solution at the heating imidation is not particularly limited but the lower limit is usually 1% by weight, preferably 5% by weight, more preferably 10% by weight as weight percent of the alicyclic polyesterimide precursor and the upper limit is 80% by weight, preferably 60% by weight, more preferably 50% by weight.

With regard to the heating temperature on this occasion, the lower limit is 100° C., preferably 120° C., more preferably 150° C. On the other hand, the upper limit can be freely set so far as it is a temperature at which no coloration of the objective compound occurs, and the solution is heated at 300° C., preferably 250° C., more preferably 200° C. On this occasion, in order to achieve azeotropic removal of water and the like which are by-products of the cyclizing imidation reaction, the reaction may be carried out with adding an azeotropic solvent such as toluene or xylene and removing water formed together with the solvent.

The reaction may be carried out with adding a basic substance as a catalyst for the cyclizing imidation reaction. Examples of the base catalyst usable in the invention include aromatic amines such as pyridine, γ-picoline, and pyrazine.

On the other hand, the chemical imidation can be carried out by adding the dehydrating reagent to the solution of the alicyclic polyesterimide precursor. The reaction is usually carried out in the presence of the dehydrating reagent and the basic substance. As the dehydrating reagent usable in the chemical imidation, there may be mentioned acid anhydrides of lower carboxylic acids such as acetic anhydride and trifluoroacetic anhydride, anhydrides of aromatic dicarboxylic acids such as trimellitic anhydride and pyromellitic anhydride, alkylcarbodiimides such as N,N-dicyclohexylcarbodiimide, and the like. On that occasion, with regard to the amount of the dehydrating reagent to be used, the lower limit is 1.0 molar equivalent, preferably 2.0 molar equivalents, more preferably 4.0 molar equivalents and the upper limit is not particularly limited and is usually 50 molar equivalents, preferably 30 molar equivalents, more preferably 20 molar equivalents to the number of mol of the amidic acid contained in the alicyclic polyesterimide precursor. There arise problems that the reaction proceeds slowly when the amount of the dehydrating reagent is too small and the reagent remains in the objective product when the amount is too large.

On the other hand, the kind of the basic substance usable is not particularly limited and organic tertiary amines such as pyridine, triethylamine, tributylamine, N,N-dimethylaniline, and dimethylaminopyridine and inorganic basic substances such as potassium carbonate and sodium hydroxide can be used. Of these, pyridine and triethylamine are preferred in view of availability in low costs and in view of easiness of reaction operations since they are liquid and rich in solubility.

With regard to the amount of the basic substance to be used, the lower limit is usually 0.1 molar equivalent, preferably 0.5 molar equivalent, more preferably 1.0 molar equivalent or more and the lower limit is usually 30 molar equivalents, preferably 20 molar equivalents, more preferably 10 molar equivalents, to the amidic acid group. There arise problems that the reaction proceeds slowly when the amount of the basic substance is too small and the substance remains in the objective product when the amount is too large. As the reaction solvent, the solvent to be used at the synthesis of the alicyclic polyesterimide precursor mentioned above can be used.

With regard to the reaction temperature to be employed, the lower limit is −10° C., preferably −5° C., more preferably 0° C. and the upper limit is 80° C., preferably 60° C., more preferably 40° C. With regard to the reaction time, the lower limit is usually 5 minutes, preferably 10 minutes and the upper limit is not particularly limited and is usually 100 hours, preferably 24 hours. The reaction is usually carried out under normal pressure but, if necessary, can be carried out under elevated pressure or under reduced pressure.

Usually, with regard to the reaction atmosphere, the reaction is carried out under nitrogen. The imidation ratio by the imidation reaction can be regulated by controlling the amount of the catalyst, the reaction temperature, and the reaction time.

The terminal amino group can be protected as an amide group by adding a reagent such as benzoyl chloride or acetic anhydride and pyridine to a solution transformed from the alicyclic polyesterimide obtained by the above process or a solution thereof obtained in the reaction. Thereby, the polyimide is prevented from coloration and its stability is increased, so that the protection is preferred.

In the process of the imidation in the presence of the dehydrating reagent and the basic substance as mentioned above, a polyesterisomide which is an isomer of the polyesterimide is sometimes mixed. The mixing ratio of the polyesterisomide is usually 90% or less, preferably 80% or less. With regard to the polyesterimide mixed with the polyesterisomide, after transformed into a powder or transformed into a film by dissolving it again in a solvent and coating a substrate therewith, the mixed polyesterisomide can be isomerized into polyesterimide by heating. With regard to the temperature on this occasion, as the lower limit, 100° C., preferably 200° C., or more preferably 300° C. can be employed. On the other hand, as the upper limit, 500° C., preferably 400° C., or more preferably 350° C. can be employed. Moreover, with regard to the reaction time on that occasion, the lower limit is usually 5 minutes, preferably 10 minutes and the upper limit is not particularly limited and is usually 100 hours, preferably 24 hours.

(ii) Process of Obtaining Alicyclic Polyesterimide without Intervening the Alicyclic Polyesterimide Precursor

As a process of obtaining the alicyclic polyesterimide without intervening the alicyclic polyesterimide precursor, it is also possible to produce the alicyclic polyesterimide of the invention by reacting the alicyclic tetracarboxylic anhydride having an ester group or a class of tetracarboxylic acid thereof represented by any of the above formulae (1) to (3) as a starting material with a class of diamine to effect a direct cyclizing imidation reaction.

The process is a process of direct cyclizing imidation without isolating in mid-course the alicyclic polyesterimide precursor which is an intermediate. As the reaction conditions on that occasion, the conditions for the heating imidation which produces the alicyclic polyesterimide from the aforementioned alicyclic polyesterimide precursor can be suitably employed.

### <Method of Converting Form of Alicyclic Polyesterimide>

When the alicyclic polyesterimide of the invention obtained as above is dissolved in a solvent to form a solution (varnish), an alicyclic polyesterimide in a variously changed form can be easily produced. For example, when it is added to a large amount of a poor solvent and filtrated, the alicyclic polyesterimide can be isolated as a powder. The poor solvent usable on this occasion is not particularly limited but there can be mentioned water, methanol, acetone, hexane, butyl cellosolve, heptane, methyl ethyl ketone, methyl isobutyl ketone, ethanol, toluene, benzene, and the like. After filtration and recovery, a specific polymer precipitated by pouring it into the poor solvent can be dried at ordinary temperature or under heating under normal pressure or under reduced pressure to form a powder. Moreover, when operations of re-dissolving the powdered alicyclic polyesterimide in an organic solvent and re-precipitating and recovering it are repeated twice to ten times, impurities in the alicyclic polyesterimide can be reduced. When three or more kinds of poor solvents such as an alcohol, a ketone, and a hydrocarbon are used as poor solvents, the efficiency of purification is further increased, so that the case is preferred.

The powdery alicyclic polyesterimide thus obtained can be re-dissolved in a solvent to form a solution (varnish).

As the solvent usable on that occasion, the solvents used at the synthesis of the alicyclic polyesterimide precursor can be used.

Furthermore, in addition to them, for the purpose of improving uniformity of a coated film, there can be also used solvents having a low surface tension, such as ethyl cellosolve, butyl cellosolve, ethylcarbitol, butylcarbitol, ethylcarbitol acetate, ethylene glycol, 1-methoxy-2-propanol, 1-ethoxy-2-propanol, 1-butoxy-2-propanol, 1-phenoxy-2-propanol, propylene glycol monoacetate, propylene glycol diacetate, propylene glycol-1-monomethyl ether-2-acetate, propylene glycol-1-monoethyl ether-2-acetate, dipropylene glycol, 2-(2-ethoxypropoxy)propanol, lactic acid methyl ester, lactic acid ethyl ester, lactic acid n-propyl ester, lactic acid n-butyl ester, and lactic acid isoamyl ester. These solvents may be used singly or as a mixture of two or more thereof.

Moreover, the mixing amount of the solvent for the purpose of improving uniformity of a coated film is preferably 10 to 80% by weight, more preferably 20 to 60% by weight in the whole solvent. With regard to the concentration of the alicyclic polyesterimide on this occasion, the lower limit is usually 1% by weight, preferably 5% by weight, more preferably 10% by weight and the upper limit is usually 80% by weight, preferably 60% by weight, more preferably 50% by weight. The alicyclic polyesterimide solution (varnish) thus obtained can be used for film formation and for coating as a coating material for various materials.

Furthermore, it is possible to remove foreign particles contained by filtrating the solution of the alicyclic polyesterimide. Removal of the foreign particles is of importance in optical applications. With regard to the amount of the foreign particles in the alicyclic polyesterimide precursor obtained in the invention, usually, insoluble fine particles having a projected area circle-corresponding diameter of 5 to 20 μm is 5,000 pieces or less, preferably 3,000 pieces or less, more preferably 1,000 pieces or less per 1 g of the precursor. The measuring method is as mentioned above.

By compression of the alicyclic polyesterimide powder of the invention under heating, a molded article of the alicyclic polyesterimide in a desired form can be formed. With regard to the heating temperature on that occasion, heating can be performed at 150° C., preferably 200° C., more preferably 250° C. as a lower limit and, on the other hand, at 450° C., preferably 400° C., more preferably 350° C. as an upper limit. Moreover, when the alicyclic polyesterimide powder once isolated is re-dissolved, for example, in the solvent used at the polymerization, it can be restored to the polyesterimide varnish.

Furthermore, when the alicyclic polyesterimide varnish is applied on a substrate and dried, an alicyclic polyesterimide film can be formed. A method for the application is not particularly limited. For example, the alicyclic polyesterimide solution can be applied in a uniform height by adding the solution onto an optical substrate such as a quartz plate, a stainless plate, or a capton film dropwise and casting the solution by rubbing over a support whose height is fixed. On this occasion, it is possible to use a device such as a doctor blade.

In addition, as the other method for the application, there may be mentioned a spraying method, a dip-coating method, a spin-coating method, a printing method, and an inkjet method but a transfer printing method is industrially widely employed in view of productivity and the method is suitably used also in the liquid crystal-aligning agent of the invention.

The coated film thus applied still contains a large amount of the solvent. Thus, the solvent is removed under heating. With regard to the temperature on that occasion, the lower limit is usually 70° C., preferably 100° C., more preferably 150° C. and the upper limit is usually 350° C., preferably 300° C., more preferably 250° C. At heating, the temperature may be elevated stepwise or continuously. With regard to the atmosphere of these steps, they may be carried out under reduced pressure or in an inert atmosphere.

The degree of reduced pressure employed in the case of performing under reduced pressure is usually 0.05 MPa or less, preferably 0.01 MPa or less, more preferably 0.001 MPa or less.

These films are patterned to form a predetermined shape as an optical component by a method such as wet etching, dry etching, or laser abrasion, if necessary. Since the thus obtained optical elements such as films and optical components using the alicyclic polyesterimide of the invention exhibit small birefringence and are colorless and transparent, the physical properties thereof are extremely good even when they are thick films.

The thickness of the alicyclic polyesterimide film at its formation can be controlled by changing the thickness of the applying solution. The lower limit is usually 0.1 μm, preferably 1 μm, more preferably 5 μm and the upper limit is usually 1,000 μm, preferably 700 μm, more preferably 500 μm.

Furthermore, since the alicyclic polyesterimide of the invention is excellent in solubility to solvent, the form can be freely processed, for example, a sheet or fibers from its solution, depending on the applications. Moreover, it is also possible to use the film not only as a single-layer one but also as a multilayer one.

Into the alicyclic polyesterimide and its precursor, additives such as an oxidation stabilizer, a filler, a silane coupling agent, a light sensitive agent, a photopolymerization initiator, and a photosensitizer can be incorporated as needed. In addition, in order to achieve physical properties required for the resin, such as improvement of strength, enhancement of thermal resistance, and decrease of water absorbability, it is also possible to mix the alicyclic polyesterimide of the invention with the other resin.

The resin to be used on that occasion is not particularly limited so far as it can be homogeneously mixed with the alicyclic polyesterimide of the invention. For example, transparent resins for optical uses, such as polyimides, polyetherimides, polyesterimides having the other composition, polyethersulfones, triacetylcellulose, polycarbonates, polyesters, poly(meth)acrylates, and polycycloolefins may be used with mixing the above polyesterimide.

### <Physical Properties of Alicyclic Polyesterimide>

With regard to the glass transition temperature Tg (° C.) of the alicyclic polyesterimide of the invention, the lower limit is usually in the range of 150° C., preferably 200° C., more preferably 250° C. and the upper limit is usually 500° C., preferably 450° C., more preferably 400° C., so that it has a high thermal resistance.

The 5% weight-loss temperature as another index showing thermal resistance is usually 350° C. or higher, preferably 400° C. or higher, more preferably 420° C. or higher in an inert atmosphere and is usually 350° C. or higher, preferably 380° C. or higher, more preferably 400° C. or higher in an air atmosphere.

Moreover, the alicyclic polyesterimide of the invention has a characteristic of high transparency. In the graph of an ultraviolet-visible light absorption spectrum measured as a polyimide film having a thickness of 30 μm, it has a characteristic that average transparency in the wavelength range of 250 to 800 nm is usually 50% or more, preferably 60% or more, more preferably 70% or more. In addition, transparency of a monochrome light of 400 nm is usually 40% or more, preferably 60% or more, more preferably 70% or more. Furthermore, cut-off wavelength is usually 350 nm or less, preferably 330 nm or less, more preferably 310 nm or less. The lower limit of the cut-off wavelength is usually 220 nm or more, preferably 250 nm or more.

The alicyclic polyesterimide of the invention has a characteristic of excellent optical isotropy and small birefringence. The birefringence is usually 0.05 or less, preferably 0.01 or less, more preferably 0.005 or less.

The pencil hardness (JIS-K5400) of the alicyclic polyesterimide of the invention is usually in the range of B to 7H, preferably in the range of H to 4H.

With regard to the refractive index of the alicyclic polyesterimide of the invention, the upper limit is usually 1.75, preferably 1.70, more preferably 0.68 and the lower limit is 1.50, preferably 1.53, more preferably 1.55. In this connection, it is well known that introduction of a fluorine atom into a resin lowers the refractive index. Also, when a fluorine atom is introduced into the alicyclic polyesterimide of the invention, the dielectric constant is lowered and in that case, the upper limit is usually 1.65, preferably 1.63, more preferably 1.60 and the lower limit is 1.45, preferably 1.48, more preferably 1.50.

The dielectric constant of the alicyclic polyesterimide of the invention at 1 MHz is usually 3.2 or less, preferably 3.0 or less, more preferably 2.9 or less. Moreover, it is well known that introduction of a fluorine atom into a resin lowers the dielectric constant. When a fluorine atom is introduced into the alicyclic polyesterimide of the invention, the dielectric constant is lowered and in that case, it is usually 3.0 or less, preferably 2.8 or less, more preferably 2.7 or less. Furthermore, the polyesterimide also has a characteristic that dielectric loss tangent has low frequency dependency in the range of 1 to 20 GHz and shows almost constant value in the range of 0.005 to 0.020 and thus the polyesterimide has an extremely excellent high frequency property.

With regard to the amount of the foreign particles contained in the alicyclic polyesterimide, usually, insoluble fine particles having a projected area circle-corresponding diameter of 5 to 20 μm is 5,000 pieces or less, preferably 3,000 pieces or less, more preferably 1,000 pieces or less per 1 g of the precursor.

The water absorbability of the alicyclic polyesterimide of the invention when immersed in water at 25° C. for 24 hours is usually 5% by weight, preferably 3% by weight, more preferably 2% by weight.

The linear thermal expansion rate of the alicyclic polyesterimide of the invention is usually 100 ppm/K or less, preferably 50 ppm/K or less, more preferably 30 ppm/K.

The polyesterimide of the invention shows high solubility to solvents. Particularly, it is well dissolved in the solvents used at the synthesis of the above alicyclic polyesterimide precursor and can be easily transformed into a solution.

The alicyclic polyesterimide of the invention has a characteristic that it is flexible and can be bent when transformed into a film and it has a high restoration property capable of being restored to a flat film when allowed to go back from the bent form. Usually, it is possible to produce a film of the alicyclic polyesterimide of the invention, which is not cracked even when bent up to 180°.

The tensile strength of the alicyclic polyesterimide of the invention as a film is usually 10 MPa or more, preferably 30 MPa or more, more preferably 50 MPa or more.

The tensile modulus of the alicyclic polyesterimide of the invention as a film is usually 0.1 GPa or more, preferably 0.5 GPa or more, more preferably 1.0 GPa or more.

With regard to the tensile elongation of the alicyclic polyesterimide of the invention as a film, the lower limit is usually 0.1%, preferably 0.5%, more preferably 1.0% and the upper limit is usually 150% or less, preferably 100% or less, more preferably 80% or less.

### <Applications>

The alicyclic polyesterimide of the invention simultaneously satisfies high glass transition temperature, low birefringence, colorlessness and transparency, and low dielectric constant and, utilizing these excellent balanced properties, can be used as a material in semiconductor fields, optical material fields, optical communication fields, display device fields, electric and electronic device fields, transportation vehicle fields, aerospace fields, and the like. For example, there may be mentioned precise optical components such as lenses and diffraction gratings, substrates for disks such as hologram, CD, MD, DVD, and optical disks, and optical adhesives in the optical material fields; substrates for LCD, supporting films for polarizing plates, transparent resin sheets, retardation films, light-diffusive films, prism sheets, adhesives for LCD, spacers for LCD, electrode substrates for LCD, transparent protective films for color filters, color filters, transparent protective films, and the like as display device applications; screens for projectors, substrates and films for plasma displays, optical filters, coating materials for organic EL, and the like as display material applications other than LCD; optical fibers, light guides, light diverging devices, light mixing devices, light switching elements, light modulating devices, light filters, wavelength dividers, light amplifiers, light attenuators, light wavelength converters in the optical communication fields and the optical element fields; insulating tapes, various laminated sheets, flexible circuit boards, adhesive films for multilayer printed circuit boards, cover films for printed circuit boards, surface protective films for semiconductor integrated circuit devices, coverings for electric wires, etc. and sealants for photosemiconductors such as flash memories, CCD, PD, and LD in the electric and electronic device fields; base polymer semiconductor coatings and underfilling agents for light sensitive polymers, such as buffer coat films, passivation films, and interlayer insulating films in the semiconductor fields; and component coatings for special aerospace components such as solar cells and heat-controlling systems as well as coverings and base film substrates for solar cells, adhesives, and the other coatings utilizing the properties of the present agent in the aerospace fields.

Of these, the alicyclic polyesterimide of the invention is suitable for use as various members for liquid crystal displays since the alicyclic polyesterimide of the invention is soluble in solvents, can be transformed into a film at low temperature by coating, and has property balance of optically transparent, high light transmittance, and extremely small birefringence, the balance being not possessed by other optical resins. For example, it is possible to utilize the polyimide as a starting resin at the manufacture of members for liquid crystal displays, such as aligning films, pressure-sensitive adhesives, polarizing plates, color filters, resin black matrix materials, and viewing angle-compensatory films.

### EXAMPLES

The following will describe the invention with reference to Examples but the invention is not limited to these Examples unless it exceeds the gist.

### 1. Measurement of Physical Properties of Monomers

### <Infrared Absorption Spectrum>

The infrared absorption spectrum of a product was measured by a KBr method using a Fourier transform infrared spectrophotometer.

### <Proton NMR Spectrum>

A product was dissolved in deuterated dimethyl sulfoxide and a proton NMR spectrum was measured using an NMR photometer of a proton resonance frequency of 400 MHz.

### <Melting Point>

Melting point was determined based on an endothermic peak of melting in the course of temperature elevation at a temperature-elevating rate of 2° C./minute in a nitrogen atmosphere on a differential scanning calorimetry apparatus.

### 2. Measurement of Physical Properties of Polymers

### <Infrared Absorption Spectrum>

The infrared absorption spectrum of the alicyclic polyesterimide precursor and the alicyclic polyesterimide thin film was measured by a transmission method using a Fourier transform infrared spectrophotometer (FT-IR5300 manufactured by JASCO Corporation).

### <Intrinsic Viscosity>

A 0.5% by weight alicyclic polyesterimide precursor solution was subjected to measurement at 30° C. using an Ostwald viscometer.

### <Glass Transition Temperature: Tg>

The glass transition temperature of the alicyclic polyesterimide film was determined based on a loss peak at a frequency of 0.1 Hz and a temperature-elevating rate of 5° C./minute by a dynamic viscoelasticity measurement using an apparatus for thermomechanical analysis (TMA4000) manufactured by Bruker AX. Alternatively, it was determined based on the baseline shift at a temperature elevation rate of 10° C./minute using a differential scanning calorimeter (DSC6220) manufactured by SII Nano-technology.

### <5% Weight-Loss Temperature: T_{d}⁵>

A temperature at the time when initial weight of the alicyclic polyesterimide film decreased by 5% was measured in the course of temperature elevation at a temperature-elevating rate of 10° C./minute in a nitrogen or air atmosphere using an apparatus for thermomechanical analysis (TG-DTA2000) manufactured by Bruker AX. The higher values thereof show that the thermal stability is high.

### <Cutoff Wavelength (Transparency)>

A visible-ultraviolet light transmittance from 200 nm to 900 nm was measured using an ultraviolet-visible spectrophotometer (V-520) manufactured by JASCO Corporation. A wavelength (cutoff wavelength) at which transmittance lowered to 0.5% or less was regarded as an index of transparency. The shorter cutoff wavelength means that the transparency of the alicyclic polyesterimide film is good.

### <Light Transmittance (Transparency)>

A light transmittance at 400 nm was measured using an ultraviolet-visible spectrophotometer (V-520) manufactured by JASCO Corporation. The higher transmittance means that the transparency of the alicyclic polyesterimide film is good.

### <Birefringence>

Using an Abbe refractometer (Abbe 4T) manufactured by Atago, refractive indices of the alicyclic polyesterimide film in parallel (nᵢₙ) and vertical (nₒᵤₜ) directions were measured on the Abbe refractometer (at a wavelength of 589 nm using sodium lump) and birefringence (Δn=nᵢₙ−nₒᵤₜ) was determined from the difference between these refractive indices.

### <Dielectric Constant>

Using an Abbe refractometer (Abbe 4T) manufactured by Atago, dielectric constant (∈) of the alicyclic polyesterimide film at 1 MHz according to the following equation was calculated based on average refractive index of the alicyclic polyesterimide film [nₐᵥ=(2nᵢₙ+nₒᵤₜ)/3]. ∈=1.1×nₐᵥ²

### <Water Absorbability>

After the alicyclic polyesterimide film (film thickness of 20 to 30 μm) vacuum-dried at 50° C. for 24 hours was immersed in water at 25° C. for 24 hours, excess water was wiped off and water absorbability (%) was determined from increase in weight.

### <Linear Thermal Expansion Coefficient: CTE>

Using an apparatus for thermomechanical analysis (TMA4000) manufactured by Bruker AX, the linear thermal expansion coefficient of the alicyclic polyesterimide film was determined as an average value in the range of 100 to 200° C. from elongation of a test piece at a load of 0.5 g/1 μm-thickness and a temperature-elevating rate of 5° C./minute by thermomechanical analysis.

### <Elastic Modulus, Elongation at Break>

Using a tensile tester (Tensilon UTM-2) manufactured by Toyo Baldwin, a tensile test (stretching rate: 8 mm/minute) was carried out on a test piece (3 mm×30 mm) of the polyimide film and elastic modulus was determined from initial slope of a stress-strain curve and elongation at break (%) was determined from elongation percentage at the time when the film is broken. The higher elongation at break means that toughness of the film is high.

### 1) Production of Hydroquinone Hydrogenated Trimellitic Acid Diester

### Example 1

Chlorination of aromatic ring-hydrogenated trimellitic anhydride was carried out as follows. Into a reaction vessel fitted with a nitrogen-inlet tube and a condenser was charged 7.93 g (40 mmol) of aromatic ring-hydrogenated trimellitic anhydride. Thereto was added 80 mL (1.1 mol) of thionyl chloride and the whole was refluxed at 80° C. for 2 hours in a nitrogen atmosphere. Thereafter, anhydrous benzene was added to the reaction solution and the solvent was removed by distillation under reduced pressure in an oil bath. Further, anhydrous benzene was added and removed by distillation to remove remaining thionyl chloride completely. The product is vacuum-dried at room temperature for 15 hours to obtain white needle-like crystals of aromatic ring-hydrogenated trimellitic anhydride chloride quantitatively.

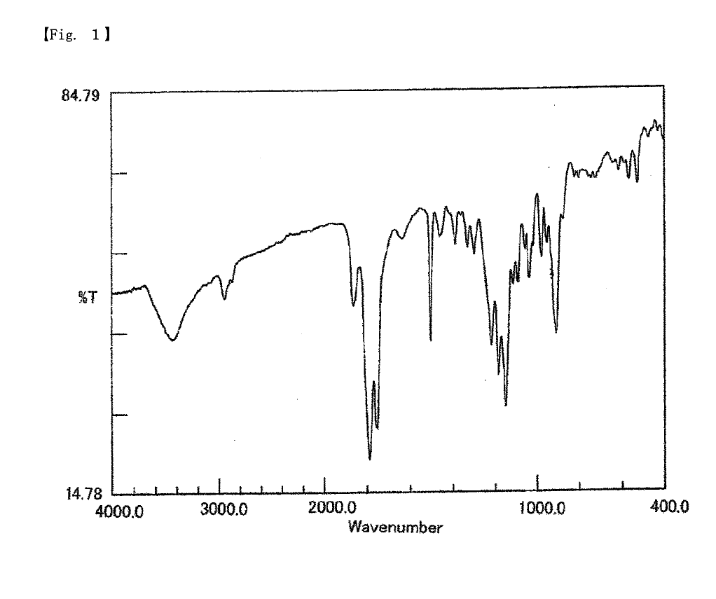

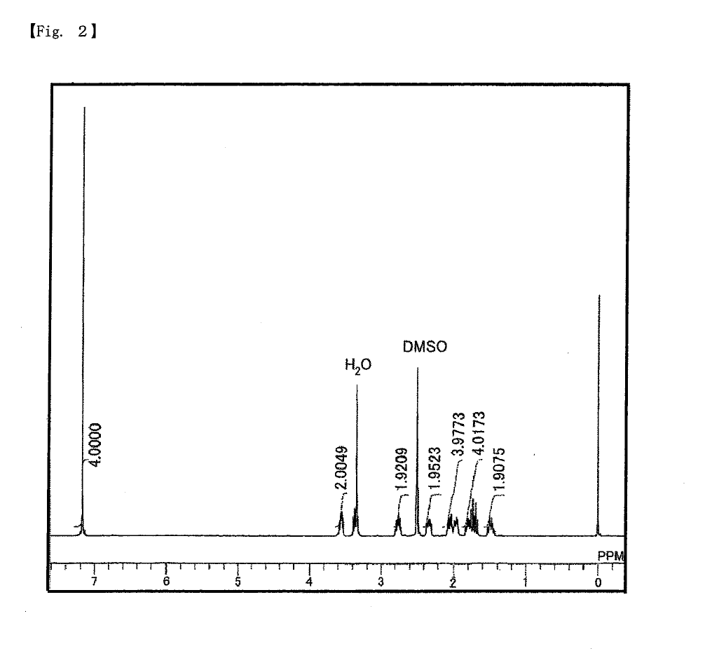

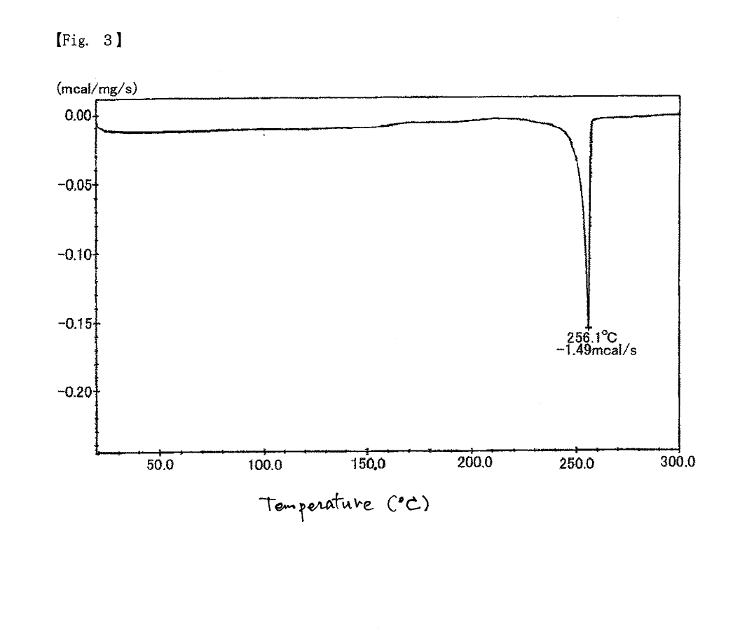

Then, 23 mL of anhydrous tetrahydrofuran was added to 8.66 g (40 mmol) of aromatic ring-hydrogenated trimellitic anhydride chloride in a reaction vessel and it was dissolved, followed by sealing with a septum cap. In another reaction vessel, 2.20 g (20 mmol) of hydroquinone and 13 mL (160 mmol) of pyridine were dissolved in 6 mL of anhydrous tetrahydrofuran, followed by sealing with a septum cap. To the solution kept at 0° C. in an ice bath, the above solution of aromatic ring-hydrogenated trimellitic anhydride chloride dissolved in anhydrous tetrahydrofuran was added dropwise by means of a syringe over a period of one hour, followed by stirring for another 9 hours to obtain a white precipitate. After separation thereof by filtration, a hydrochloride was completely removed by thorough washing with water and the product was vacuum-dried at 150° C. for 20 hours to obtain a white power in 83% yield. The compound showed a sharp endothermic peak (melting point: 256° C.) by differential scanning calorimetry. Moreover, from infrared spectrum and proton NMR spectrum, it was confirmed that the resulting product was an objective alicyclic tetracarboxylic dianhydride having a structure of the following formula (9). The results are shown in FIG. 1 to FIG. 3. In addition, the structure of the hydroquinone hydrogenated trimellitic acid diester obtained in Example 1 is shown in the following formula (9).

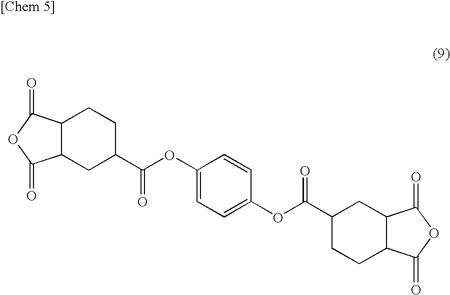

### 2) Production of Alicyclic Polyesterimide Starting from Hydroquinone Hydrogenated Trimellitic Acid Diester

### Example 2

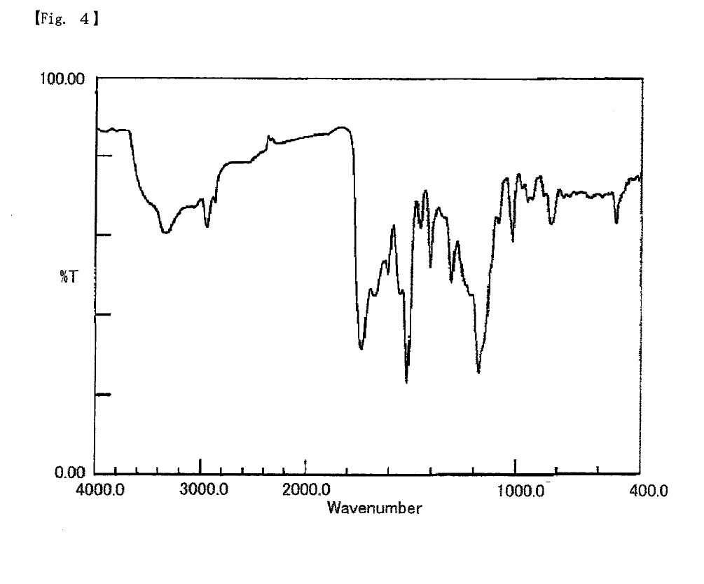

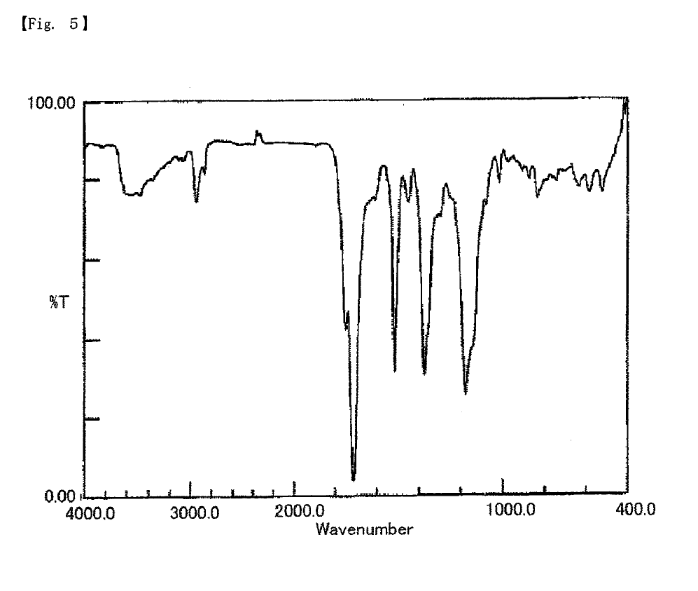

In a well-dried tightly closed reaction vessel fitted with a stirrer, 1.08 g (10 mmol) of p-phenylenediamine was dissolved in 19.3 g of N,N-dimethylacetamide. To the solution was gradually added 4.70 g (10 mmol) of the tetracarboxylic dianhydride powder produced in Example 1, followed by stirring at room temperature for 22 hours to obtain a transparent viscous alicyclic polyesterimide precursor solution. Polymerization was started at a solute concentration of 30% by weight and the reaction was carried out with adding the solvent in mid-course, finally the solution being diluted to 17% by weight. The alicyclic polyesterimide precursor solution showed extremely high solution storage stability with no occurrence of precipitation and gelation even when the solution was left on standing at room temperature and at −20° C. for one month. The intrinsic viscosity of the alicyclic polyesterimide precursor measured at 30° C. in N,N-dimethylacetamide was 1.34 dL/g and it was an extremely high polymer. The alicyclic polyesterimide precursor solution was applied on a glass substrate and dried at 60° C. for 2 hours to obtain an alicyclic polyesterimide precursor film. An infrared absorption spectrum of the resulting alicyclic polyesterimide precursor film is shown in FIG. 4. The precursor film was subjected to heat treatment on the substrate at 320° C. for 1 hour under reduced pressure and imidation was effected to obtain an alicyclic polyesterimide film. In order to remove residual strain, the film was peeled from the substrate and further subjected to heat treatment at 235° C. just below the glass transition temperature for 1 hour to obtain a transparent film having a film thickness of 30 μm. An infrared absorption spectrum of the film is shown in FIG. 5. The film was not broken by a 180° bending test and showed toughness. With regard to the film physical properties, the film showed relatively high thermal resistance of glass transition temperature of 253° C. and extremely high transparency of a cutoff wavelength of 312 nm and a transmittance at 400 nm of 72.1%.

Moreover, the resin showed a very low value of birefringence of Δn=0.0002 and hence was found to be suitable for optical materials. The dielectric constant was a relatively low value of 2.83. Furthermore, the resin showed a high solubility to organic solvents such as N-methyl-2-pyrrolidone, N,N-dimethylacetamide, N,N-dimethylformamide, and m-cresol at room temperature and workability was found to be good. As the other physical properties, water absorbability was 3.1%, 5% weight-loss temperature was 424° C. in nitrogen and 412° C. in the air, linear thermal expansion coefficient was 70.1 ppm/K, elastic modulus was 1.2 GPa, and elongation at break was 4.3%. The structure of the resulting polyesterimide obtained in Example 2 is shown in the following formula (10).

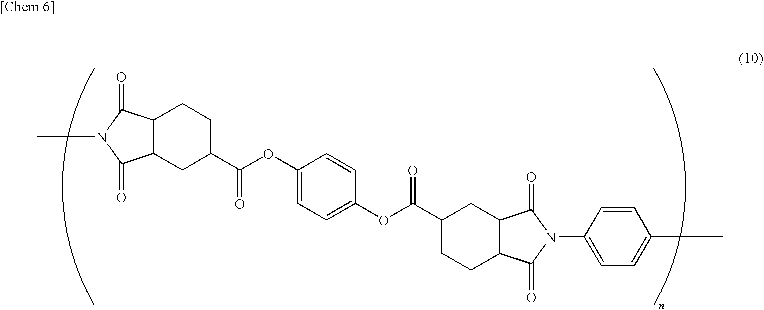

### Example 3

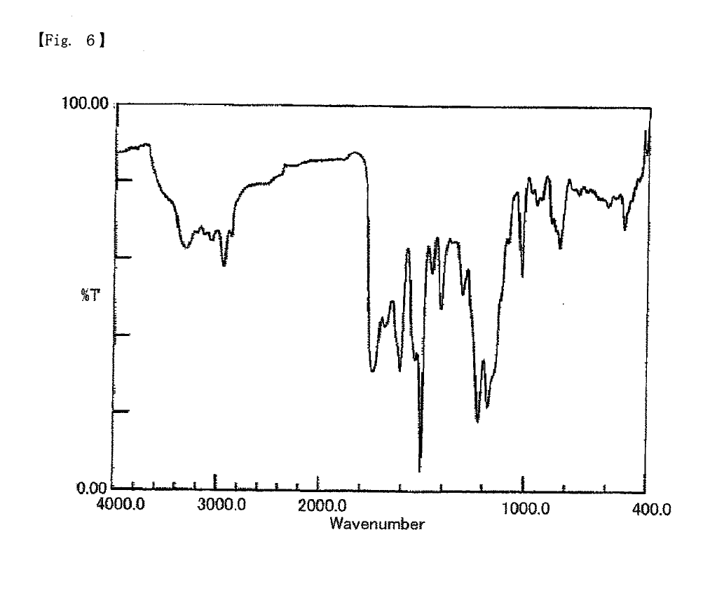

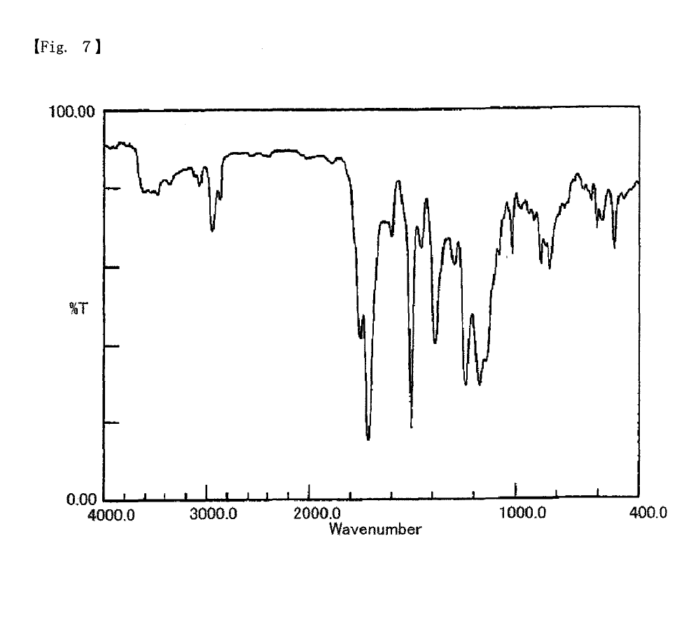

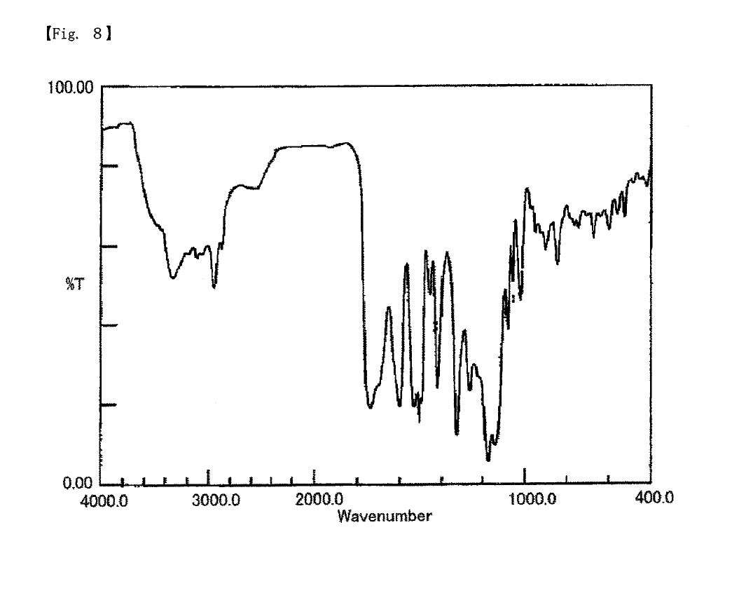

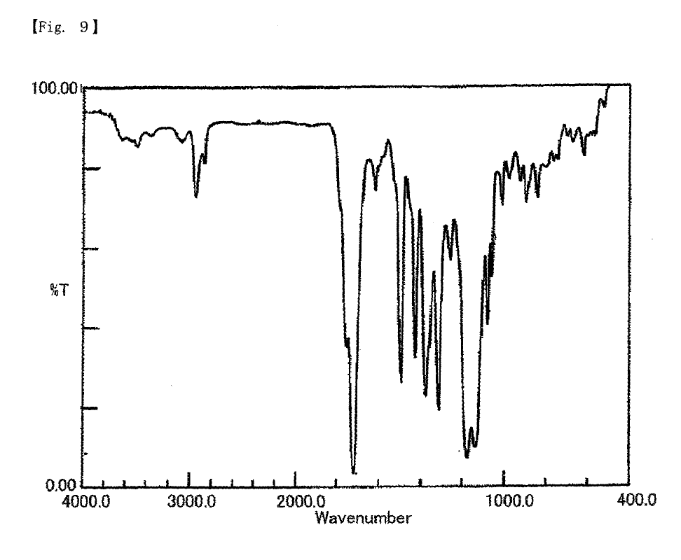

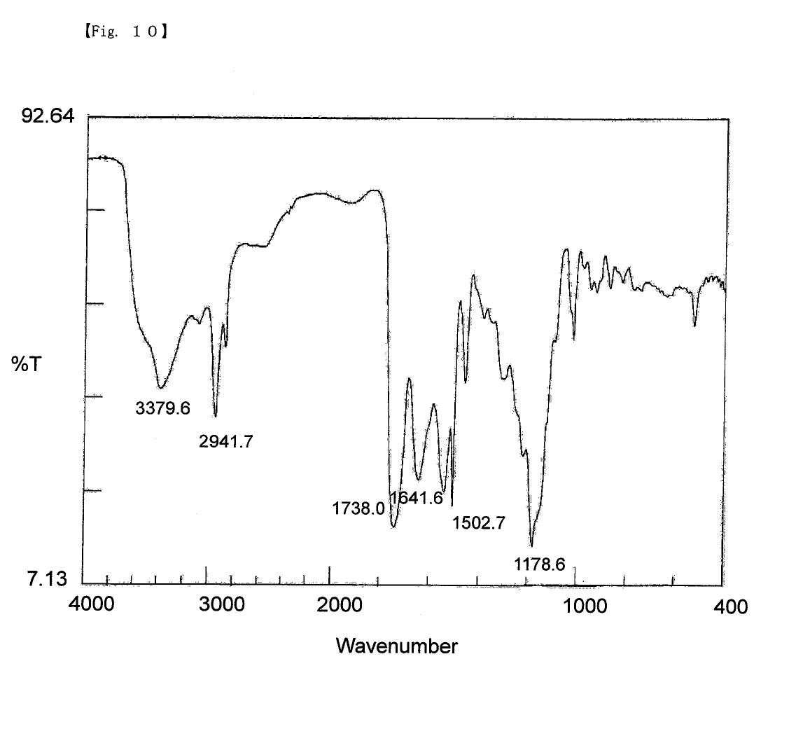

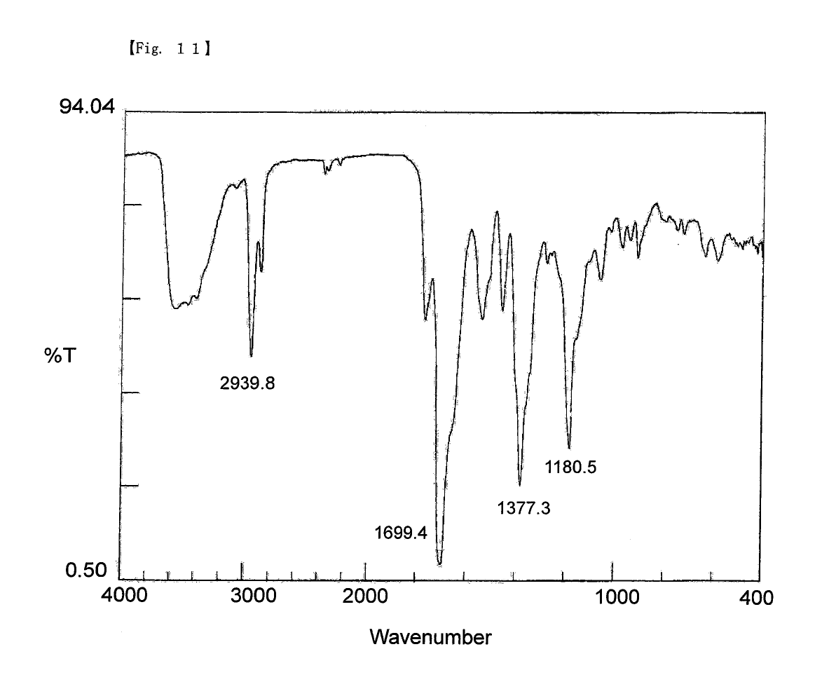

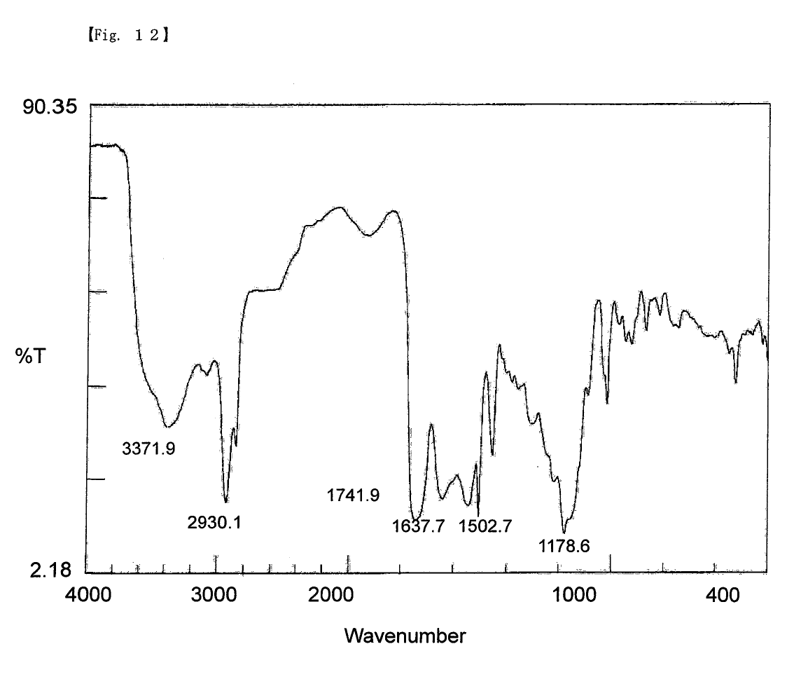

In a well-dried tightly closed reaction vessel fitted with a stirrer, 2.00 g (10 mmol) of 4,4′-oxydianiline was dissolved in 22.3 g of N,N-dimethylacetamide. To the solution was gradually added 4.70 g (10 mmol) of the tetracarboxylic dianhydride powder produced in Example 1, followed by stirring at room temperature for 22 hours to obtain a transparent viscous alicyclic polyesterimide precursor solution. Polymerization was started at a solute concentration of 30% by weight, finally the solution being diluted to 13% by weight. The alicyclic polyesterimide precursor solution showed extremely high solution storage stability with no occurrence of precipitation and gelation even when the solution was left on standing at room temperature and at −20° C. for one month. The intrinsic viscosity of the alicyclic polyesterimide precursor measured at 30° C. in N,N-dimethylacetamide was 2.32 dL/g and it was an extremely high polymer. The alicyclic polyesterimide precursor solution was applied on a glass substrate and dried at 60° C. for 2 hours to obtain an alicyclic polyesterimide precursor film. An infrared absorption spectrum of the resulting alicyclic polyesterimide precursor film is shown in FIG. 6. The precursor film was subjected to heat treatment on the substrate at 320° C. for 1 hour under reduced pressure and imidation was effected to obtain an alicyclic polyesterimide film. In order to remove residual strain, the film was peeled from the substrate and further subjected to heat treatment at 218° C. just below the glass transition temperature for 1 hour to obtain a transparent film having a film thickness of 30 μm. An infrared absorption spectrum of the film is shown in FIG. 7. The film was not broken by a 180° bending test and showed toughness. With regard to the film physical properties, the film showed relatively high thermal resistance of glass transition temperature of 225° C. and extremely high transparency of a cutoff wavelength of 301 nm and a transmittance at 400 nm of 81.3%. The birefringence of the resin was very small as Δn=0.0005 and hence the resin was found to be suitable for optical materials. The dielectric constant was a relatively low value of 2.83. Furthermore, the resin showed a high solubility to organic solvents such as N-methyl-2-pyrrolidone, N,N-dimethylacetamide, N,N-dimethylformamide, and m-cresol at room temperature and workability was found to be good. As the other physical properties, water absorbability was 1.1%, 5% weight-loss temperature was 428° C. in nitrogen and 418° C. in the air, and linear thermal expansion coefficient was 76.4 ppm/K. The structure of the polyesterimide obtained in Example 3 is shown in the following formula (11).

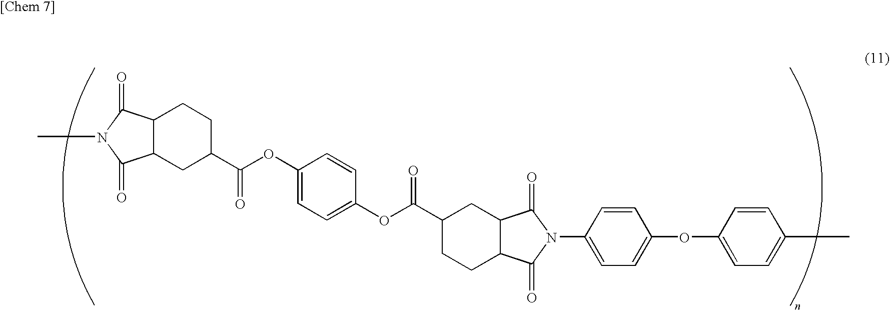

### Example 4

In a well-dried tightly closed reaction vessel fitted with a stirrer, 3.20 g (10 mmol) of 2,2′-bis(trifluoromethyl)-4,4′-diaminobiphenyl was dissolved in 22.3 g of N,N-dimethylacetamide. To the solution was gradually added 4.70 g (10 mmol) of the tetracarboxylic dianhydride powder produced in Example 1, followed by stirring at room temperature for 22 hours to obtain a transparent viscous alicyclic polyesterimide precursor solution. Polymerization was started at a solute concentration of 30% by weight, finally the solution being diluted to 19% by weight. The alicyclic polyesterimide precursor solution showed extremely high solution storage stability with no occurrence of precipitation and gelation even when the solution was left on standing at room temperature and at −20° C. for one month. The intrinsic viscosity of the alicyclic polyesterimide precursor measured at 30° C. in N,N-dimethylacetamide was 1.29 dL/g and it was an extremely high polymer. The alicyclic polyesterimide precursor solution was applied on a glass substrate and dried at 60° C. for 2 hours to obtain an alicyclic polyesterimide precursor film. The precursor film was subjected to heat treatment on the substrate at 350° C. for 1 hour under reduced pressure and imidation was effected to obtain an alicyclic polyesterimide film. In order to remove residual strain, the film was peeled from the substrate and further subjected to heat treatment at 235° C. just below the glass transition temperature for 1 hour to obtain a transparent film having a film thickness of 30 μm. The film was not broken by a 180° bending test and showed toughness. With regard to the film physical properties, the film showed relatively high thermal resistance of glass transition temperature of 250° C. and extremely high transparency of a cutoff wavelength of 304 nm and a transmittance at 400 nm of 80.1%. The birefringence of the resin was very small as Δn=0.002 and hence the resin was found to be suitable for optical materials. The dielectric constant was an extremely low value of 2.67. Furthermore, the resin showed a high solubility to organic solvents such as N-methyl-2-pyrrolidone, N,N-dimethylacetamide, N,N-dimethylformamide, and m-cresol at room temperature and workability was found to be good. As the other physical properties, water absorbability was 1.29%, 5% weight-loss temperature was 441° C. in nitrogen and 407° C. in the air, and linear thermal expansion coefficient was 82.1 ppm/K. The structure of the polyesterimide obtained in Example 4 is shown in the following formula (12).

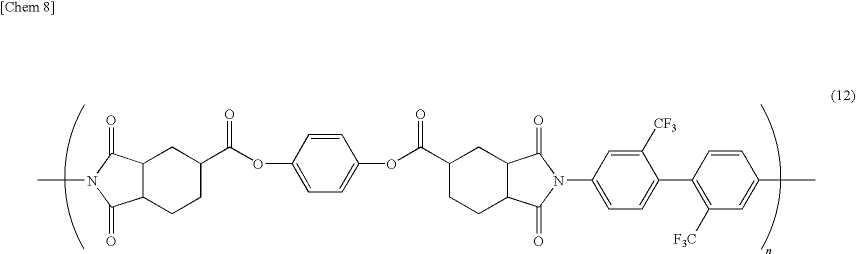

### Example 5

An alicyclic polyesterimide film was obtained in the same manner except that the diamine in Example 2 was changed to t-1,4-cyclohexanediamine (10 mmol). The intrinsic viscosity of thereof in mid-course was 1.15 dL/g and it was an extremely high polymer. With regard to the film physical properties, the film showed relatively high thermal resistance of glass transition temperature of 243° C. and extremely high transparency of a cutoff wavelength of 263 nm and a transmittance at 400 nm of 70.0%. The birefringence of the resin was very small as Δn=0.0011 and hence the resin was found to be suitable for optical materials. The dielectric constant was an extremely low value of 2.70. As the other physical properties, 5% weight-loss temperature was 408° C. in nitrogen and 399° C. in the air and linear thermal expansion coefficient was 90.8 ppm/K. The structure of the polyesterimide obtained in Example 5 is shown in the following formula (13).

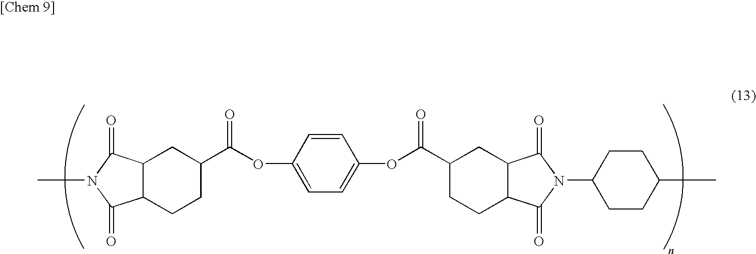

### Example 6

An alicyclic polyesterimide film was obtained in the same manner except that the diamine in Example 2 was changed to t,t-methylenebiscyclohexylamine (10 mmol). The intrinsic viscosity of thereof in mid-course was 1.20 dL/g and it was an extremely high polymer. With regard to the film physical properties, the film showed relatively high thermal resistance of glass transition temperature of 210° C. and extremely high transparency of a cutoff wavelength of 271 nm and a transmittance at 400 nm of 68.2%. The birefringence of the resin was very small as Δn=0.00012 and hence the resin was found to be suitable for optical materials. The dielectric constant was an extremely low value of 2.63. Furthermore, the resin showed a high solubility to organic solvents such as N-methyl-2-pyrrolidone, N,N-dimethylacetamide, N,N-dimethylformamide, and m-cresol at room temperature and workability was found to be good. As the other physical properties, 5% weight-loss temperature was 412° C. in nitrogen and 391° C. in the air and linear thermal expansion coefficient was 75.0 ppm/K. The structure of the polyesterimide obtained in Example 6 is shown in the following formula (14).

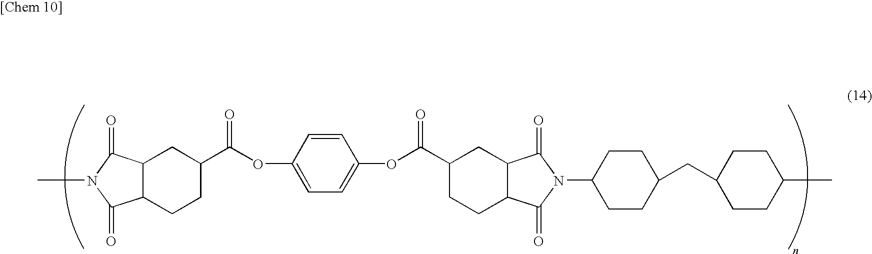

### Example 7

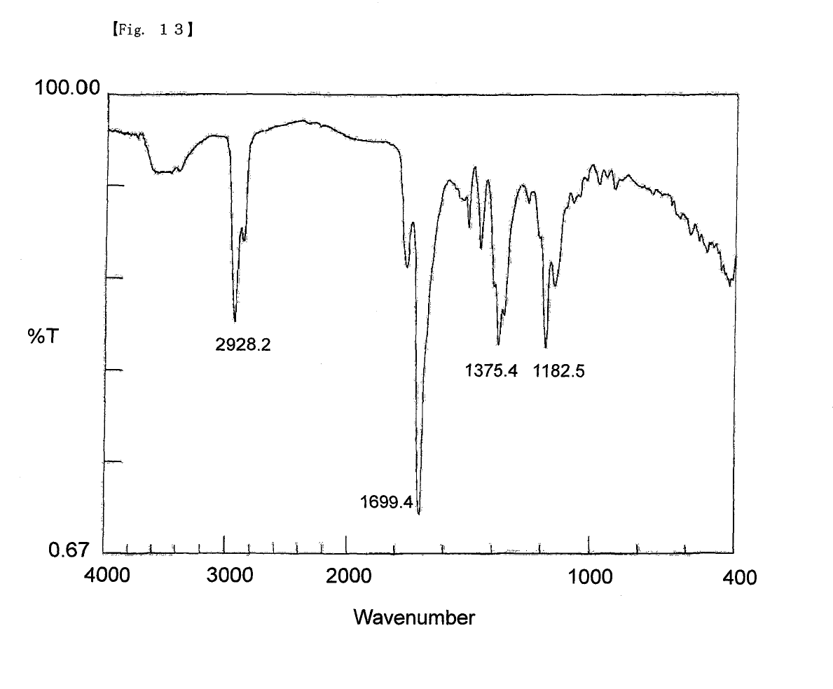

In a 50 mL three-neck flask, 0.400 g (3.70 mmol) of p-phenylenediamine was dissolved in 8.19 g of N,N-dimethylacetamide. To the solution was gradually added 1.76 g (3.74 mmol) of the tetracarboxylic dianhydride powder produced in Example 1, followed by stirring at room temperature for 14 hours to obtain a transparent viscous alicyclic polyesterimide precursor solution. Polymerization was started at a solute concentration of 26% by weight, finally the solution being diluted to 13% by weight (intrinsic viscosity: 1.53 dL/g). Thereafter, it was diluted with 9.40 g of N,N-dimethylacetamide and further 2.34 g of pyridine and 4.91 g of acetic anhydride were added thereto, followed by stirring at 50° C. for 7 hours. The content was added to 150 ml of methanol and precipitated solid was filtrated, washed with methanol, and vacuum-dried at 100° C. to obtain 1.65 g of a polyesterimide powder. For film formation, the synthesized polyesterimide powder was dissolved in NMP (about 15% by weight) and the solution was applied on a glass substrate. After drying at 80° C. for 1 hour, heat treatment was performed at 200° C. for 1 hour under reduced pressure and a film was peeled from the glass substrate to obtain a transparent film having a film thickness of 20 μm. An infrared absorption spectrum of the film is shown in FIG. 14. With regard to the film physical properties of the resulting alicyclic polyesterimide film, the film showed relatively high thermal resistance of glass transition temperature of 230° C. (value measured by DSC) and extremely high transparency of a cutoff wavelength of 275 nm and a transmittance at 400 nm of 86.2%. The structure of the polyesterimide obtained in the present Example is the same as the formula (10) of Example 2.

### Example 8

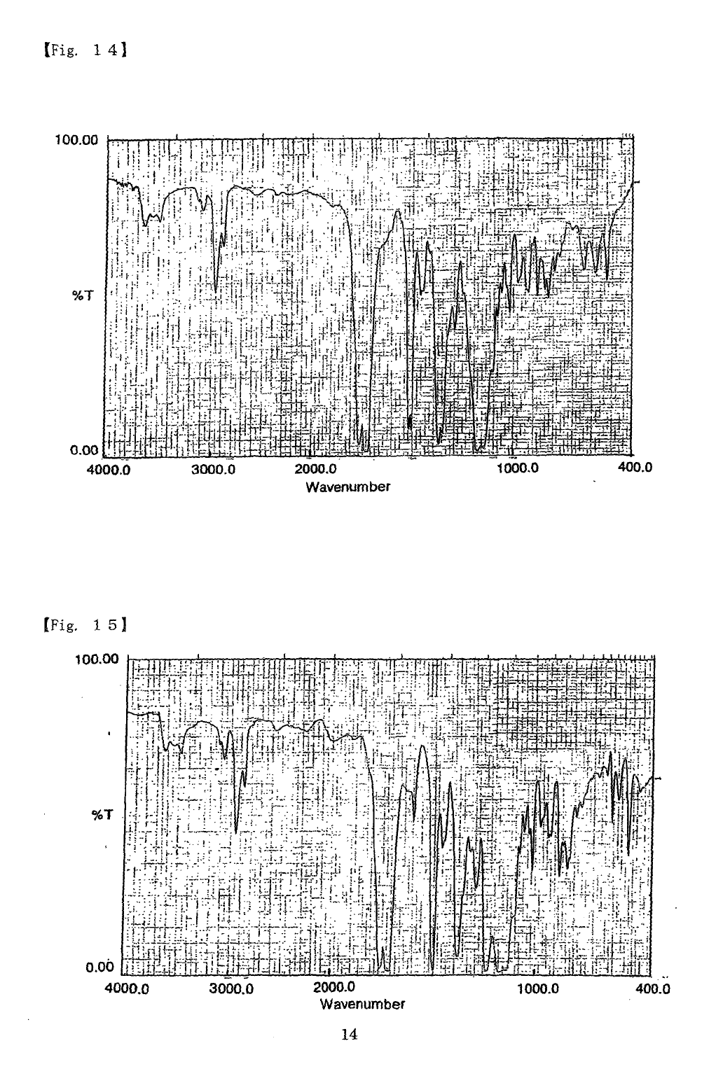

An alicyclic polyesterimide film was obtained in the same manner as in Example 7 except that the diamine used is changed to 4,4′-oxydianiline. An infrared absorption spectrum of the film is shown in FIG. 15. With regard to the film physical properties of the resulting alicyclic polyesterimide film, the film showed relatively high thermal resistance of glass transition temperature of 207° C. (value measured by DSC) and extremely high transparency of a cutoff wavelength of 289 nm and a transmittance at 400 nm of 88.0%. The structure of the polyesterimide obtained in the present Example is the same as the formula (11) of Example 3.

### 3) Production of Hydrogenated Trimellitic Acid Diester of 1,4-hexanediol

### Example 9

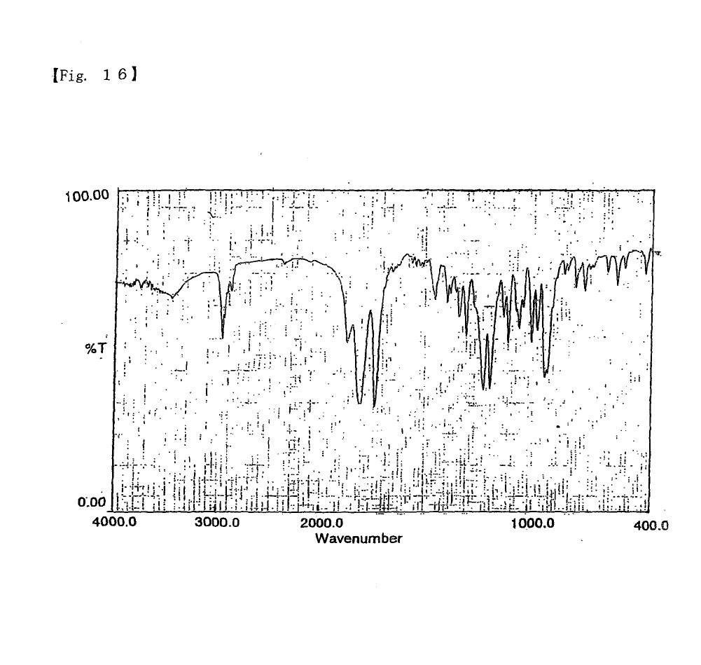

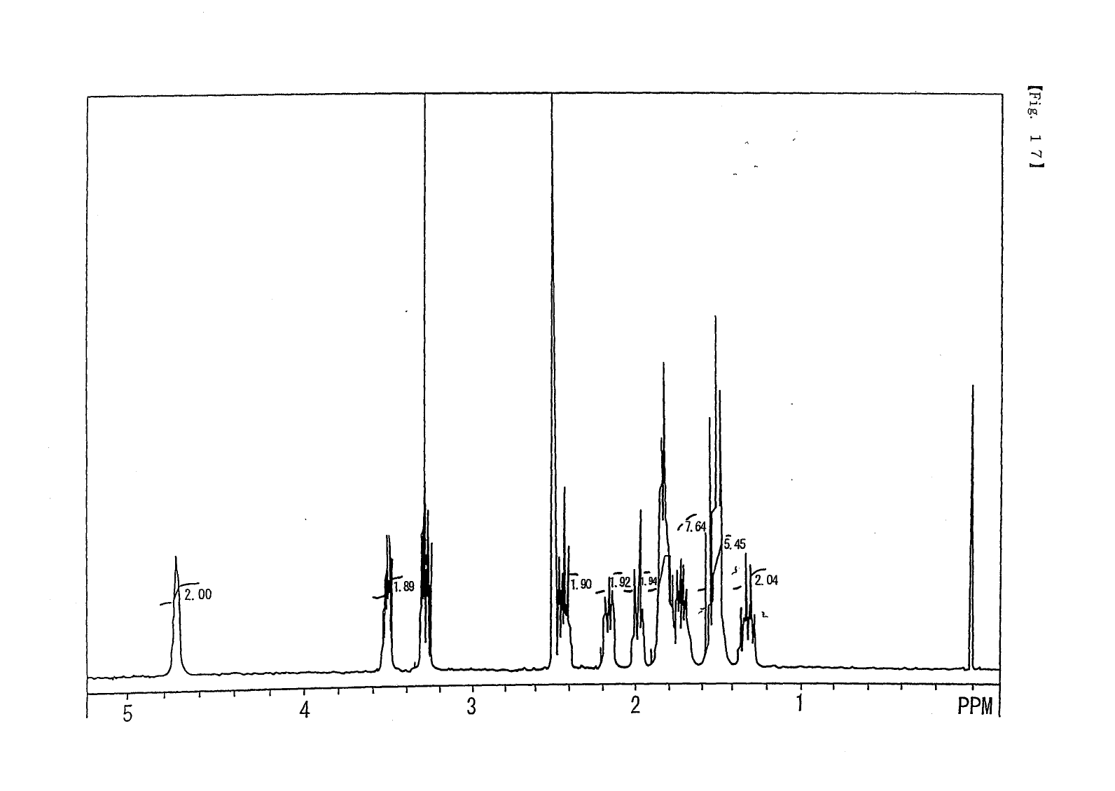

Ten mL of tetrahydrofuran was added to 4.99 g (23.1 mmol) of aromatic ring-hydrogenated trimellitic anhydride chloride and it was dissolved. Moreover, 1.31 g (11.3 mmol) of 1,4-cyclohexanediol and 1.82 g (23.1 mmol) of pyridine were dissolved in 15 mL of tetrahydrofuran. To the solution kept at 4° C. in an ice bath, the above solution of aromatic ring-hydrogenated trimellitic anhydride chloride dissolved in tetrahydrofuran was added dropwise over a period of 15 minutes, followed by stirring at room temperature for another 16 hours. After the precipitated white precipitate was separated by filtration, it was thoroughly washed with water and dried at 100° C. for 5 hours under reduced pressure to obtain 1.97 g of a white solid. After the solid was recrystallized from 25 ml of acetic anhydride/acetic acid (2/3 in volume ratio), it was vacuum-dried at 150° C. for 7 hours to obtain 0.88 g (yield 16.4%) of a white powder. The compound showed a sharp endothermic peak (melting point: 238° C.) by differential scanning calorimetry. Moreover, from infrared spectrum and proton NMR spectrum, it was confirmed that the resulting product was an objective alicyclic tetracarboxylic dianhydride having a structure of the following formula (15). The results are shown in FIG. 16 and FIG. 17. The structure of the 1,4-hexanediol hydrogenated trimellitic acid diester obtained in the present Example is shown in the following formula (15).

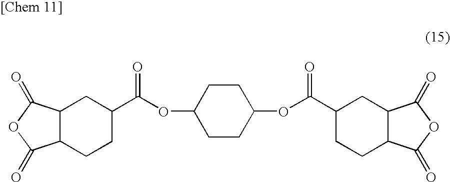

### 4) Production of Alicyclic Polyesterimide Starting from Acid Dianhydride Represented by Above Formula (15)

### Example 10

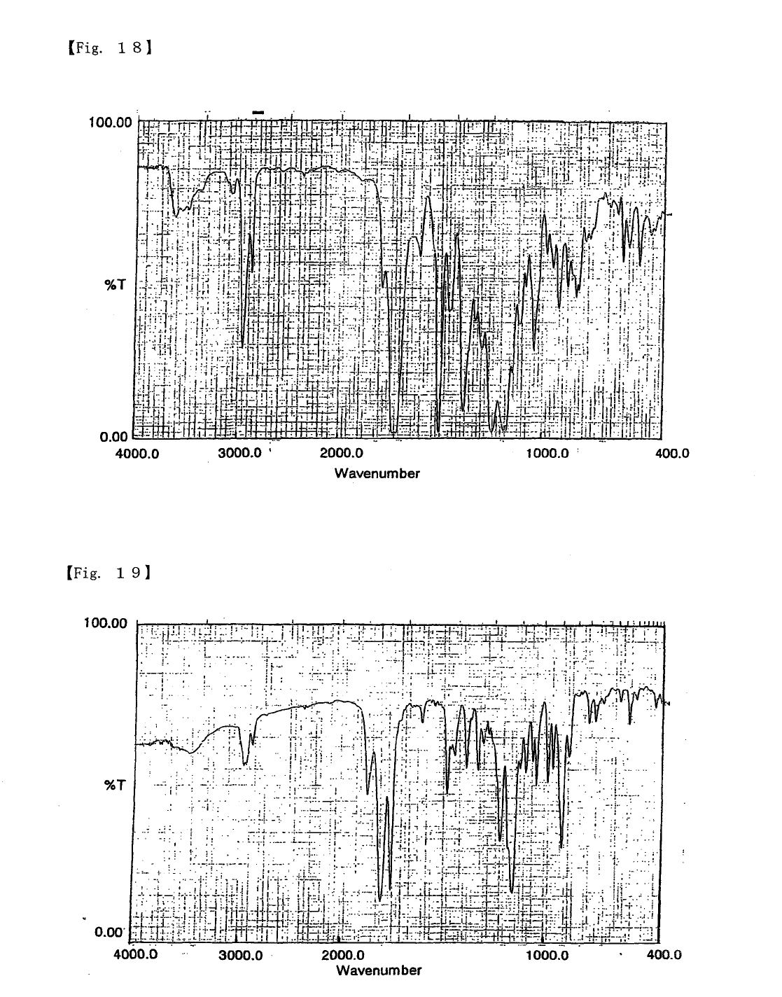

A polyesterimide film was obtained in the same manner as in Example 7 except that the tetracarboxylic dianhydride used was changed to one produced in Example 9 and the diamine used is changed to 4,4′-oxydianiline. Furthermore, film formation of the resulting polyesterimide was performed in the same manner as in Example 7 except that m-cresol was used as a dissolution solvent to obtain an alicyclic polyesterimide film. An infrared absorption spectrum of the film is shown in FIG. 18. With regard to the film physical properties of the resulting alicyclic polyesterimide film, the film showed relatively high thermal resistance of glass transition temperature of 164° C. (value measured by DSC) and extremely high transparency of a cutoff wavelength of 288 nm and a transmittance at 400 nm of 85.3%. The structure of the polyesterimide obtained in Example 10 is shown in the following formula (16).

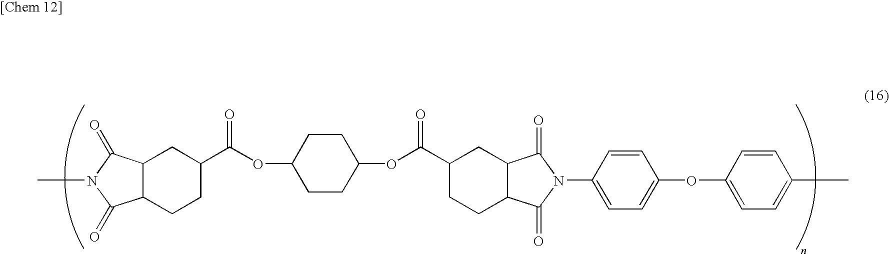

### 5) Production of Hydrogenated Trimellitic Acid Diester of 3,3′,5,5′-tetramethylbiphenyl-4,4′-diol

### Example 11

Ten mL of tetrahydrofuran was added to 5.04 g (23.1 mmol) of aromatic ring-hydrogenated trimellitic anhydride chloride and it was dissolved. Moreover, 2.74 g (11.3 mmol) of 3,3′,5,5′-tetramethylbiphenyl-4,4′-diol and 1.82 g (23.1 mmol) of pyridine were dissolved in 15 mL of tetrahydrofuran. To the solution kept at 4° C. in an ice bath, the above solution of aromatic ring-hydrogenated trimellitic anhydride chloride dissolved in tetrahydrofuran was added dropwise over a period of 10 minutes, followed by stirring at room temperature for another 16 hours. After the precipitated white precipitate was separated by filtration, it was thoroughly washed with water and then vacuum-dried at 150° C. for 7 hours to obtain 5.52 g (yield 81.2%) of a white powder. The compound showed a sharp endothermic peak (melting point: 329° C.) by differential scanning calorimetry. Moreover, from infrared spectrum and proton NMR spectrum, it was confirmed that the resulting product was an objective alicyclic tetracarboxylic dianhydride having a structure of the following formula (17). The results are shown in FIG. 19. The structure of the hydroquinone hydrogenated trimellitic acid diester obtained in Example 11 is shown in the following formula (17).

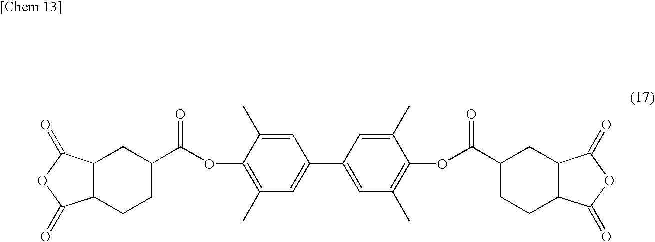

### 6) Production of Alicyclic Polyesterimide Starting from Acid Dianhydride Represented by Above Formula (17)

### Example 12

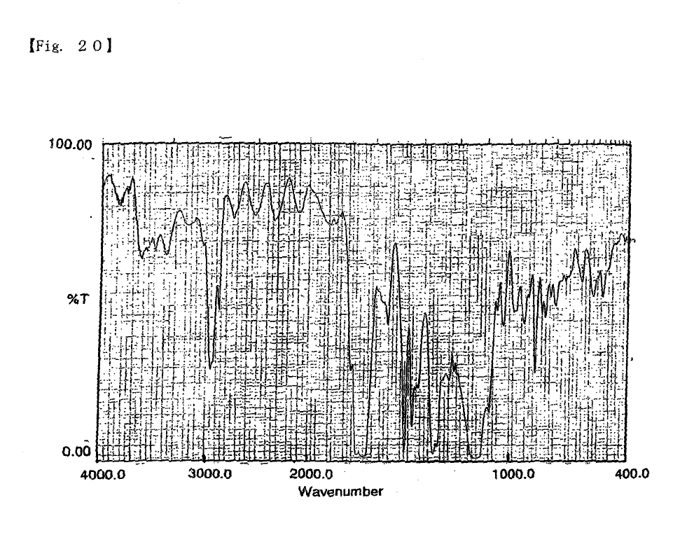

A polyesterimide film was obtained in the same manner as in Example 7 except that the tetracarboxylic dianhydride used was changed to one produced in Example 11 and the diamine used is changed to p-phenylenediamine. Furthermore, film formation of the resulting polyesterimide was performed in the same manner as in Example 7 to obtain an alicyclic polyesterimide film. An infrared absorption spectrum of the film is shown in FIG. 20. With regard to the film physical properties of the resulting alicyclic polyesterimide film, the film showed relatively high thermal resistance of glass transition temperature of 255° C. (value measured by DSC) and extremely high transparency of a cutoff wavelength of 299 nm and a transmittance at 400 nm of 74.3%. The structure of the polyesterimide obtained in Example 12 is shown in the following formula (18).

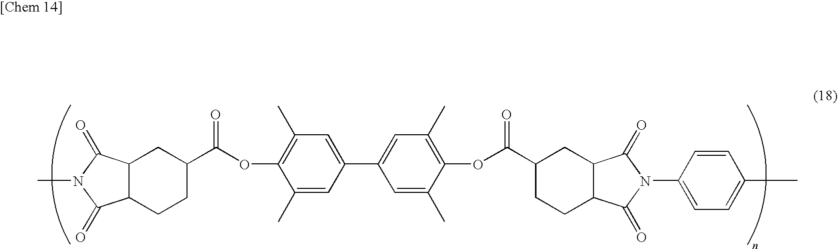

### 7) Production of Hydrogenated Trimellitic Acid Diester of 4,4′-(9-fluorenylidene)diphenol

### Example 13

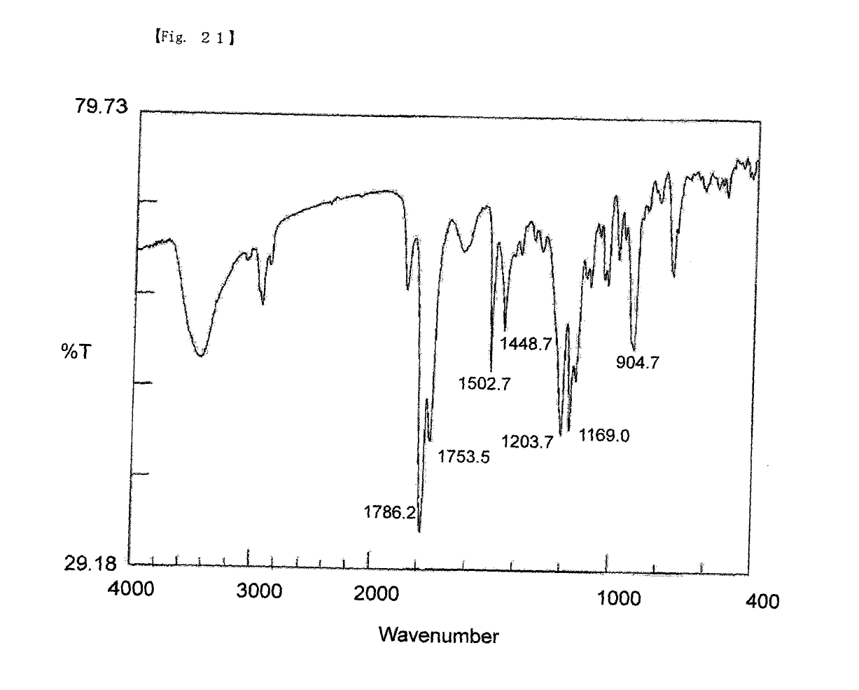

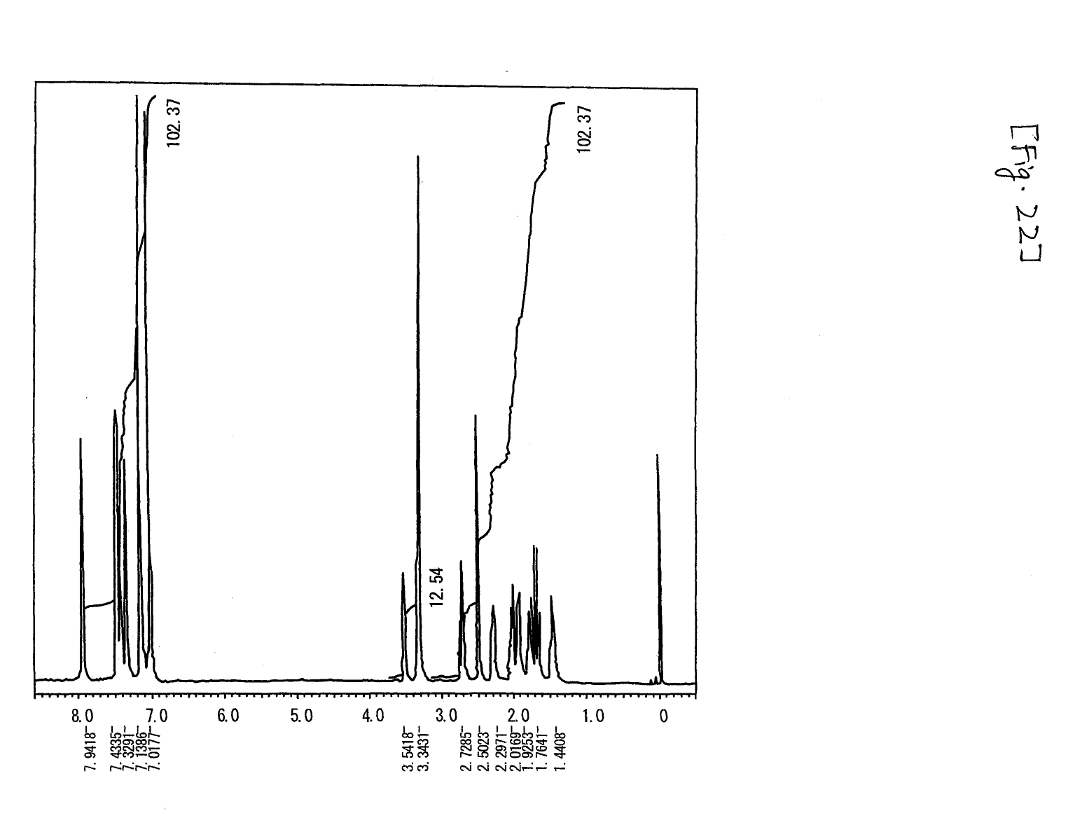

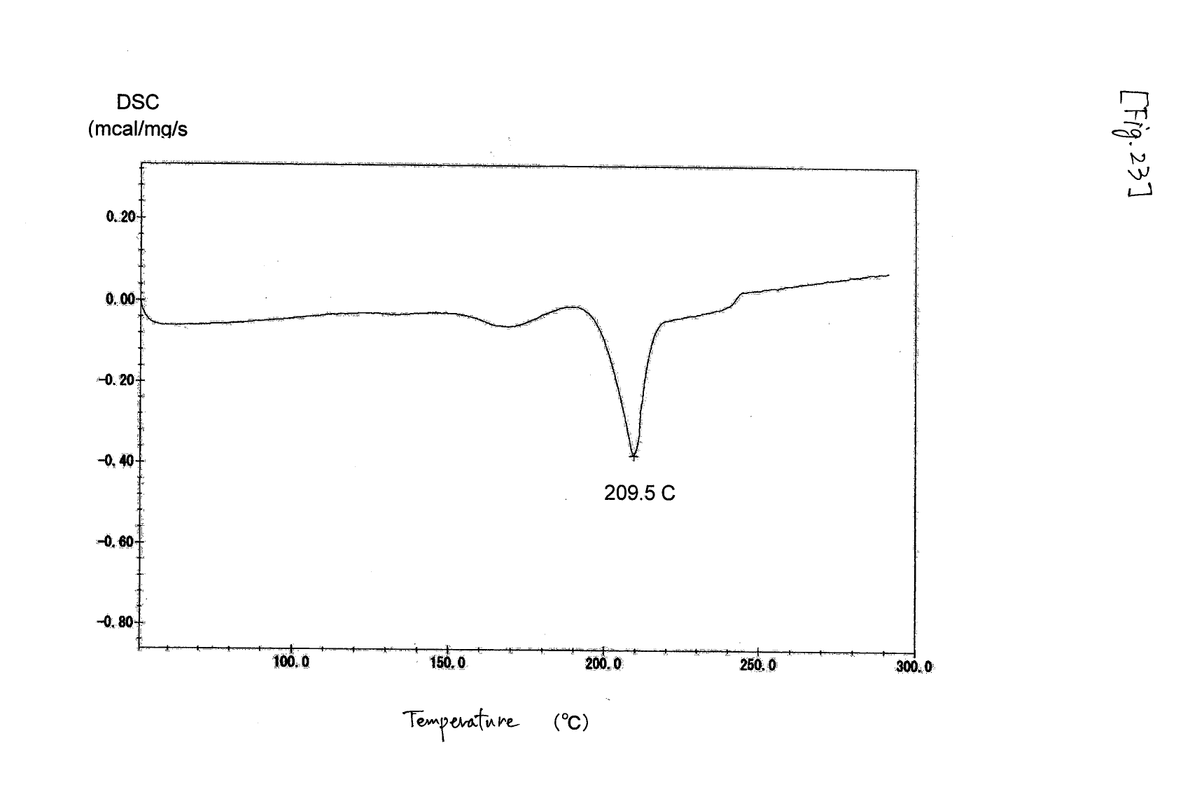

In a reaction vessel, 15 mL of tetrahydrofuran was added to 4.33 g (20 mmol) of aromatic ring-hydrogenated trimellitic anhydride chloride and it was dissolved, followed by sealing with a septum cap. In another reaction vessel, 3.51 g (10 mmol) of 9,9-bis(4-hydroxyphenyl)fluorene and 3.24 mL (40 mmol) of pyridine were dissolved in 12 mL of anhydrous tetrahydrofuran, followed by sealing with a septum cap. To the solution kept at 0° C. in an ice bath, the above solution of aromatic ring-hydrogenated trimellitic anhydride chloride dissolved in anhydrous tetrahydrofuran was added dropwise by means of a syringe over a period of one hour, followed by stirring at room temperature for another 24 hours to obtain a white precipitate. After separation thereof by filtration, a hydrochloride was removed and the filtrate was subjected to solvent removal by distillation on an evaporator. Finally, the resulting product was vacuum-dried at 120° C. for 24 hours to obtain a white power in 89.3% yield. The compound showed an endothermic peak (melting point: 209.5° C.) by differential scanning calorimetry. Moreover, from infrared spectrum and proton NMR spectrum, it was confirmed that the resulting product was an objective alicyclic tetracarboxylic dianhydride containing a fluorenyl group, which has a structure represented by the following formula (19). The results are shown in FIG. 21 to FIG. 23. In addition, the structure of the tetracarboxylic anhydride containing a fluorenyl group obtained in Example 7 is shown in the following formula (19).

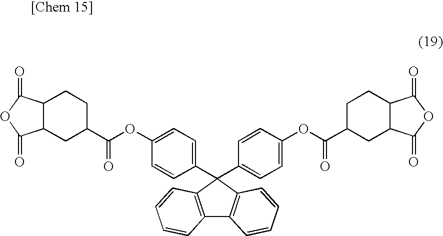

### Comparative Example

In a well-dried tightly closed reaction vessel fitted with a stirrer, 1.08 g (10 mmol) of p-phenylenediamine was placed and then dissolved in 15 mL of N,N-dimethylacetamide. Thereafter, to the solution was gradually added 4.58 g (10 mmol) of an aromatic tetracarboxylic dianhydride powder corresponding to the tetracarboxylic dianhydride powder described in Example 1. Since the solution viscosity rapidly increased, the solution was suitably diluted with the solvent and, after hour, 52 mL was added for dilution. The whole was further stirred for 24 hours to obtain a transparent, homogeneous, and viscous aromatic polyesterimide precursor solution. The intrinsic viscosity of the aromatic polyesterimide precursor measured at 30° C. in N,N-dimethylacetamide in a concentration of 0.5% by weight was 5.19 dL/g. The aromatic polyesterimide precursor solution was applied on a glass substrate and dried at 60° C. for 2 hours to obtain an aromatic polyesterimide precursor film. After the film was subjected to thermal imidation at 250° C. for 2 hours under reduced pressure on the substrate, it was peeled from the substrate in order to remove residual stress and was further subjected to heat treatment at 350° C. for 1 hour to obtain an aromatic polyesterimide film having a film thickness of 20 μm. The aromatic polyesterimide film did not show any solubility to any organic solvents. When film physical properties were measured, glass transition temperature was not detected until 450° C. Moreover, cutoff wavelength was 369 nm and transmittance at 400 nm was 22%, so that transparency was remarkably low as compared with the alicyclic polyesterimide described in Example 2. This is attributed to large absorption in a UV region since the aromatic tetracarboxylic dianhydride containing an ester group is used as a monomer. The birefringence of the resin was so extremely large as Δn=0.219 and thus it was found to be entirely not suitable for optical materials. The dielectric constant was a relatively high value of 3.22. As the other physical properties, water absorbability was 1.4% and 5% weight-loss temperature was 480.7° C. in nitrogen and 463.2° C. in the air. The structure of the resulting polyesterimide obtained in Comparative Example is shown in the following formula (20).

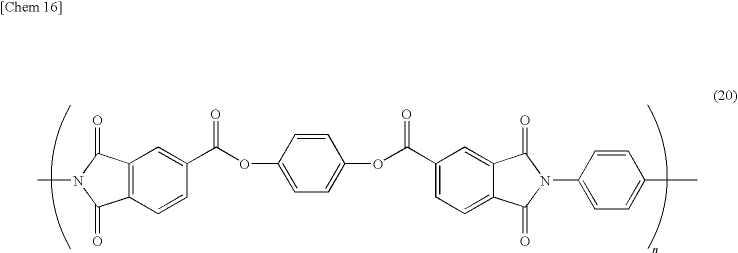

While the invention has been described in detail and with reference to specific embodiments thereof, it will be apparent to one skilled in the art that various changes and modifications can be made therein without departing from the spirit and scope thereof. The present application is based on Japanese Patent Application No. 2005-161490 filed on Jun. 1, 2005, Japanese Patent Application No. 2005-264852 filed on Sep. 13, 2005 and Japanese Patent Application No. 2006-081058 filed on Mar. 23, 2006, and the contents are incorporated herein by reference.

### INDUSTRIAL APPLICABILITY

According to the present invention, there can be provided a resin having all of high glass transition temperature, high transparency, high organic solvent solubility, low birefringence, and alkali-etching properties in combination as well as a starting material thereof. Specifically, owing to the bonding of the acid anhydride group onto the cyclohexane ring in the tetracarboxylic dianhydride which is a starting material of the resin according to the invention, enhancement of transparency and decrease in dielectric constant become possible by suppressing n-electron conjugation and intramolecular and intermolecular charge transfer interaction in the polyesterimide. Moreover, the ester bond in the polyesterimide enables alkali-etching in the case where micro-fabrication such as through-hole formation is necessary.

1. An alicyclic tetracarboxylic anhydride having an ester group or a class of tetracarboxylic acid thereof, which is represented by any of the following general formulae (1) to (3): 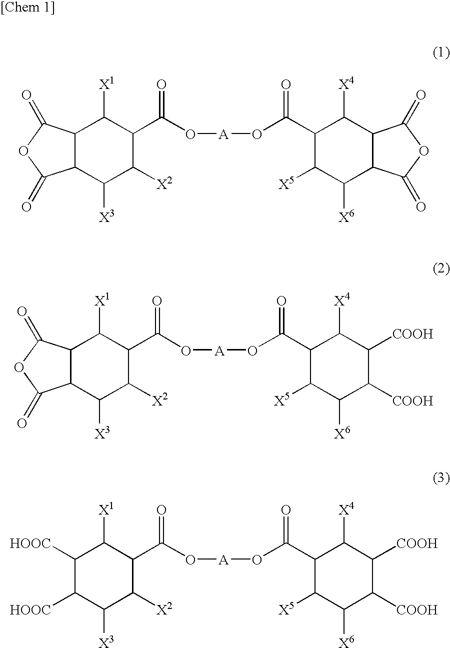
wherein in the above formulae (1) to (3), A represents a divalent group and X¹, X², X³, X⁴, X⁵, and X⁶ each independently represents a hydrogen atom, a halogen atom, a nitrile group, a nitro group, an alkyl group, an alkenyl group, an alkynyl group, an alkoxy group, an amino group, or an amide group.

2. The alicyclic tetracarboxylic anhydride having an ester group or a class of tetracarboxylic acid thereof according to claim 1, wherein A in the above formulae (1) to (3) is a divalent group having an aromatic group and/or an aliphatic group.

3. The alicyclic tetracarboxylic anhydride having an ester group or a class of tetracarboxylic acid thereof according to claim 1, wherein in the above formulae (1) to (3), X¹, X², X³, X⁴, X⁵, X⁶, and X is a hydrogen atom and A is a structure containing at least one cyclic structure.

4. A process for producing the alicyclic tetracarboxylic anhydride having an ester group or a class of tetracarboxylic acid thereof according to claim 3, which comprises: converting an aromatic ring-hydrogenated trimellitic anhydride into an acid halide; and reacting the resulting acid halide with a diol in the presence of a basic substance.

5. An alicyclic polyesterimide precursor containing a constitutional unit represented by the following general formula (4): 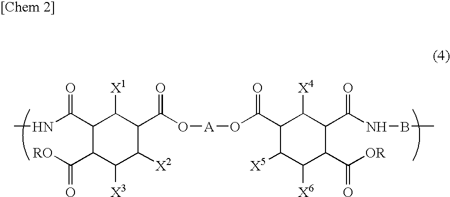
wherein in the above formula (4), A represents a divalent group; X¹, X², X³, X⁴, X⁵, and X⁶ each independently represents a hydrogen atom, a halogen atom, a nitrile group, a nitro group, an alkyl group, an alkenyl group, an alkynyl group, an alkoxy group, an amino group, or an amide group; B represents a divalent aromatic or aliphatic group; and R represents a hydrogen atom, an alkyl group having 1 to 10 carbon atoms, or a silyl group.

6. An alicyclic polyesterimide containing a constitutional unit represented by the general formula (5): 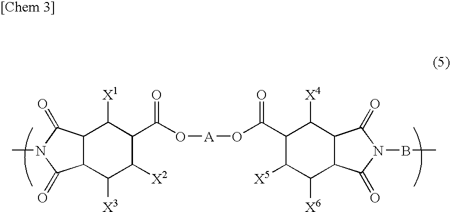
wherein in the above formula (5), A represents a divalent group; X¹, X², X³, X⁴, X⁵, and X⁶ each independently represents a hydrogen atom, a halogen atom, a nitrile group, a nitro group, an alkyl group, an alkenyl group, an alkynyl group, an alkoxy group, an amino group, or an amide group; and B represents a divalent aromatic or aliphatic group.

7. A process for producing the alicyclic polyesterimide according to claim 6, which comprises: reacting the alicyclic tetracarboxylic dianhydride containing an ester group according to any one of claims 1 to 3 with a class of diamine; and subsequently subjecting the resulting product to a cyclizing imidation reaction.

8. A process for producing the alicyclic polyesterimide according to claim 6, wherein the alicyclic polyesterimide precursor according to claim 5 is subjected to a cyclizing imidation reaction.

9. The process for producing the alicyclic polyesterimide according to claim 7 or 8, wherein the cyclizing imidation reaction is carried out using heating and/or a dehydrating reagent.

10. A film produced from a resin containing the constitutional unit of the general formula (5) according to claim 6.

11. A member for liquid crystals using the film according to claim 10.
